(19) United States
(12) Patent Application Publication
(10) Pub. No.: US 2017/0253886 A1
(43) Pub. Date: Sep. 7, 2017

(54) PLANTS WITH REDUCED ETHYLENE SENSITIVITY

(71) Applicants: Københavns Universitet, Copenhagen K (DK); Gartneriet PKM A/S, Odense N (DK)

(72) Inventors: Renate Petra Brigitte Müller, Malmö (SE); Henrik Vik Lütken, Brønshøj (DK); Josefine Nymark Hegelund, Skibby (DK); Line Jensen, København NV (DK); Andreas Olsen, Valby (DK); Christian Hald Madsen, Korsør (DK)

(21) Appl. No.: 15/506,452

(22) PCT Filed: Aug. 26, 2015
(86) PCT No.: PCT/DK2015/050249

§ 371 Date: Feb. 24, 2017

(30) Foreign Application Priority Data
Aug. 26, 2014 (DK) PA 2014 70509
Aug. 29, 2014 (DK) PA 2014 70524

(51) Int. Cl. C12N 15/82; C07K 14/415

(52) U.S. Cl. CPC C12N 15/8249; C07K 14/415

(57) ABSTRACT

The present invention relates to ethylene insensitive EIN/EIL polypeptides, and nucleotide sequences encoding the EIN/EIL polypeptides. Further, the invention relates to plants having reduced ethylene insensitivity and methods for preparing these. Lastly, the invention relates to products and crops from plants as well as processed products derived from the products and crops.

### FIELD OF THE INVENTION

The present invention in a first aspect relates to EIN/EIL polypeptides for reducing ethylene sensitivity in a plant. In further aspects, the invention relates to plants having reduced ethylene sensitivity and methods for preparing these. Lastly, the invention relates to products and crops from these plants as well as processed products derived from the products and crops.

### PRIOR ART

Ethylene is a gaseous phytohormone involved in regulating processes encompassing flower development, fruit ripening, promotion of leaf and flower senescence and abscission, dormancy release, and cell elongation (Ables et al., 1992; Mudge, 1988) as well as leaf curling and adventitious root formation (Drew et al., 1979). Ethylene responses are negatively regulated by a receptor gene family in Arabidopsis thaliana, cf. J Hua, E M Meyerowitz— Cell, 1998. Further, ethylene is involved in abscission of leaves, buds, flowers, and petals of certain plants (Müller et al., 2001a,b, 2011; Sriskandarajah et al., 2004). In the ethylene response pathway, the hormone is perceived by receptor proteins integrated in the endoplasmatic reticulum (ER) where ethylene binds to the ethylene receptors via a copper (I) co-factor (Cu⁺) (Bleecker et al.; 1988; Hua et al., 1998; and Voet-van-Vormizeele and Groth, 2008; Rodriguez, et al., 1999). The receptor proteins cloned from Arabidopsis belong to two subfamilies: subfamily 1 comprising ethylene receptor 1 (ETR1) and ethylene response sensor 1 (ERS1), and subfamily 2 consisting of ethylene receptor 2 (ETR2), ethylene insensitive 4 (EIN4) and ethylene response sensor 2 (ERS2) (Hua et al., 1998). At low ethylene levels, the receptors (ETR1, ERS1, ETR2, EIN4, and ERS2) suppress ethylene signalling by activating the negative regulator constitutive triple response 1 (CTR1) by direct physical interaction (Clarke et al., 1998). CTR1 is a Raf-like mitogen-activated protein kinase kinase kinase (MAPKKK), acting downstream of receptors and upstream of the positive regulator of ethylene-ethylene insensitive 2 (EIN2) (Kieber et al., 1993; Roman et al., 1995; Alonso et al., 1999). When CTR1 is deactivated, it relieves the repression of EIN2, leading to activation of ethylene insensitive 3/ethylene insensitive-like 1 (EIN3/EIL1)-dependent transcription and the activation of the ethylene response (Qiao et al., 2012).

Ethylene receptors have been the main target of genetic approaches focusing on reduced ethylene sensitivity against both endogenous and exogenous ethylene. In Arabidopsis, a mutated ETR1 gene, named etr1-1, fails to bind ethylene (Bleecker et al., 1988; Chang et al., 1993). When the etr1-1 was isolated from Arabidopsis (Wilkinson et al., 1997; Clarke et al., 1998) and introduced into Petunia (Clevenger et al., 2004; Gubrium et al., 2000) and Campanula carpatica (Sriskandarajah et al., 2004), transformed plants became ethylene insensitive, exhibited delayed flower senescence, and postponed flower abscission. Ethylene insensitive Petunia has also been obtained using a mutated ers homologue from Brassica oleracea. Like etr1-1, this gene encodes an ethylene receptor that does not bind ethylene (Shaw et al., 2002). In petunia transformed with etr1-1 or RNAi PhEIN2, the expression of PhEIL1, a petunia homologue of Arabidopsis EIN3 which was induced by ethylene in wild type plants, was significantly reduced. The plants transformed with RNAi PhEIN2 also exhibited delayed flower senescence (Shibuya et al., 2004).

Low amounts of endogenous and/or exogenous ethylene can cause or accelerate flower senescence. The factors affecting endogenous ethylene production comprise stress, increased CO₂ production (Finlayson and Reid, 1994), increased auxin production (increases ethylene production) (Grossmann and Hansen, 2001), increased cytokinin production (delays ethylene production) (Chang et al., 2003) and engine combustion (OECD SIDS; Alberta Environment, 2003). Hence, ornamental plants are often treated with chemicals to improve postharvest quality and prolong flower longevity. The compound silver thiosulfate (STS) is frequently used as postharvest treatment to avoid negative effects of ethylene. However, silver (Ag) is a heavy metal and Ag(I) compounds are toxic for humans and harmful for the environment (Nell, 1992) (Beyer, 1976).

Thus, there is a continuous need to improve the methods for reducing the sensitivity of plants towards ethylene, e.g. such as to mitigate the damages caused in plants by exposure to ethylene.

### DISCLOSURE OF THE INVENTION

The present invention provides a novel nucleic acid sequence encoding for a polypeptide, wherein plants comprising the nucleic acid sequence have reduced sensitivity towards ethylene. Accordingly, in a first aspect the present invention relates to an EIN/EIL polypeptide, wherein
the EIN/EIL polypeptide at least comprises a polypeptide motif and a downstream part,
the polypeptide motif consisting of the amino acid sequence SxLy,
x being any natural amino acid, preferably A or S, and
y being any natural amino acid, preferably F, G, I or M,
and
the downstream part of the EIN/EIL polypeptide, having below 50% identity to SEQ ID NO: 10.

The polypeptide motif consisting of the amino acid sequence, SxLy, divides the EIN/EIL polypeptide in an upstream part and a downstream part.

It has surprisingly been found that the plant Campanula medium, known by the vernacular name Canterbury Bells, comprises a gene, which encodes an EIL2 polypeptide having an atypical polypeptide motif of SALI and a downstream part being highly different than corresponding downstream parts from other plants. Thus, plants endowed with genes comprising a nucleic acid sequence encoding the EIN/EIL polypeptides according to the invention will exhibit improved tolerance against ethylene.

In a particular embodiment of the present invention, the downstream part of the EIN/EIL polypeptide has below 50%, preferably below 40%, preferably below 30%, preferably below 20%, more preferred below 15%, and more preferred below 10% identity to the downstream part of the EIN/EIL polypeptide, according to SEQ ID NO: 10.

In a particular embodiment of the present invention, the EIN/EIL polypeptide comprises a polypeptide motif, wherein the downstream part of the EIN/EIL polypeptide having at least 70% identity to SEQ ID NO: 3, SEQ ID 11, SEQ ID NO: 12, SEQ ID 13, SEQ ID NO: 14.

In another embodiment, the downstream part of the EIN/EIL polypeptide may vary in length. Preferably, the downstream part of the EIN/EIL polypeptide consist of more than 15 amino acids, more than 25 amino acids, preferably more than 35 amino acids, or even more preferably more than 40 amino acids.

In another embodiment of the present invention, the downstream part of the EIN/EIL polypeptide has at least 70%, preferably at least 80%, preferably at least 85%, preferably at least 90%, more preferred at least 95%, and more preferred at least 99% identity to the downstream part the EIN/EIL2 polypeptide or part thereof, according to SEQ ID NO: 3, SEQ ID NO: 11, SEQ ID NO: 12, SEQ ID NO: 13, or SEQ ID NO: 14.

In a particular embodiment of the present invention, the upstream part of the EIN/EIL polypeptide has at least 70%, preferably at least 80%, preferably at least 85%, preferably at least 90%, more preferred at least 95%, and more preferred at least 99% identity to the upstream part the EIN/EIL2 polypeptide, according to SEQ ID NO: 3, SEQ ID NO: 11, SEQ ID NO: 12, SEQ ID NO: 13, SEQ ID NO: 14, or SEQ ID NO: 19.

In another embodiment, the upstream part of the EIN/EIL polypeptide may vary in length. Preferably, the upstream part of the EIN/EIL polypeptide consist of more than 25 amino acid, preferably more than 50 amino acid, preferably more than 75 amino acid, more preferably more than 100 amino acid, and even more preferably more than 125 amino acid. In a specific embodiment the CmEIL2 has 184 amino acids upstream of the SALI motif.

In yet another embodiment of the invention, the polypeptide motif consisting of the amino acid sequence SxLy, may be modified. Hence, serine, S, or leucine, L, may be modified with another amino acid.

Thus, in one embodiment, the polypeptide motif consisting of the amino acid sequence SxLy may be modified or substituted with one, two, three or four other amino acids. Preferably, the polypeptide motif may be SALM, SALI, SALG, SALF, SSLM, SSLI, SSLG, SSLM. Serine or Leucine, which may be present in the polypeptide motif may be modified or substituted with another amino acid.

In a second aspect of the invention, an isolated nucleic acid sequence, wherein the isolated nucleic acid sequence encodes the EIN/EIL polypeptide according to the invention or a part thereof, is provided. The isolated nucleic acid sequence having at least 70% identity to SEQ ID NO: 4, SEQ ID NO: 15, SEQ ID NO: 16, SEQ ID NO: 17, or SEQ ID NO: 18, or any part of the downstream part thereof. The downstream part of SEQ ID NO: 4, SEQ ID NO: 15, SEQ ID NO: 16, SEQ ID NO: 17, and SEQ ID NO: 18 encode the downstream part of the EIN/EIL polypeptide.

In a particular embodiment of the present invention, the isolated nucleic acid sequence having an identity to SEQ ID NO: 4, SEQ ID NO: 15, SEQ ID NO: 16, SEQ ID NO: 17, SEQ ID NO: 18, or the upstream or downstream part thereof, of at least 70%, preferably at least 80%, preferably at least 85%, preferably at least 90%, more preferred at least 95%, and more preferred at least 99% identity to the upstream part or downstream part of the EIN/EIL2 polypeptide, according to SEQ ID NO: 3, SEQ ID NO: 11, SEQ ID NO: 12, SEQ ID NO: 13, SEQ ID NO: 14, or 19. The downstream part of SEQ ID NO: 4, SEQ ID NO: 15, SEQ ID NO: 16, SEQ ID NO: 17, and SEQ ID NO: 18, encode the downstream part of the EIN/EIL polypeptide.

In yet a further aspect the present invention relates to a method for preparing a plant cell having reduced sensitivity to ethylene, comprising:
identifying a EIL2 ortholog gene in a plant cell; and
disrupting or modifying the EIL2 ortholog gene in the plant cell to obtain a gene or nucleic acid sequence that encodes a polypeptide,
optionally by introducing the nucleic acid sequence of the invention into the plant cell.

In yet another aspect, the present invention relates to a plant cell, wherein the plant cell is obtained from the group of plants comprising crops, vegetables, fruits and flowers, including climacteric fruits and climacteric flowers. Climacteric plant species are sensitive to exogenous ethylene, and as a consequence exhibit accelerated petal wilting and fruit ripening when exposed to it. Thus, according to the invention, it is particularly advantageous to make these plant species less sensitive to ethylene.

In a specific embodiment, the plant cell is obtained from the group of plants comprising Campanula, Kalanchoë, orchids, Petunia, carnation and roses.

In yet another embodiment, the plant cell is selected from the group consisting of protoplasts, gamete producing cells, and cells which regenerate into a whole plant.

According to a further aspect, the invention relates to a method for preparing a plant or a transgenic plant having reduced sensitivity to ethylene, comprising producing plants from plant cells according to the above; and selecting plants that have a reduced sensitivity to ethylene.

In a further aspect, the invention relates to a plant or a transgenic plant comprising a nucleic acid sequence encoding the EIN/EIL polypeptide according to the first aspect of the invention.

According to a final aspect is provided a product or a processed product thereof, derived from said plant or transgenic plant. Said product may originate from or be constituted by any part or organ of the plant, be it reproductive or vegetative.

### BRIEF DESCRIPTION OF THE FIGURES

In the following, the invention will be explained in greater detail with the aid of examples and with reference to non-limiting schematic drawings, which are intended to illustrate the invention.

FIG. 1: Temporal flower senescence in Campanula plants upon ethylene exposure. Graphical representation of the physiological development of Campanula portenschlagiana White GET MEE®, C. portenschlagiana Dark GET MEE®, C. portenschlagiana Blue GET MEE®, C. formanekiana Blue MARY MEE® and C. medium Sweet MEE® during ethylene exposure. Plants were exposed to concentrations of 0, 0.05 and 0.1 μL/L ethylene for 72 h and monitored every 24 hours. Plants were monitored in respect to senescence of flowers measured as either mean number of senesced flowers±SE, or % senesced flowers. The number of young flowers (1 day open flowers), left column, and older flowers (4 days open flowers), right column. The experiment was repeated twice. n=15 flowers per treatment of young and old flowers in C. portenschlagiana White GET MEE®, C. portenschlagiana Dark GET MEE®, C. portenschlagiana Blue GET MEE®, C. formanekiana Blue MARY MEE® where each plant (3 per treatment) had 5 flowers labelled. In C. medium Sweet MEE® n=between 9-12. Each plant (3 per treatment) had between 1 and 5 flowers labelled. The Y-axis represents average number of senescent flowers.

FIG. 2: Protein alignment using fragments of putative ERS2 proteins identified in Campanula. As a reference Arabidopsis ERS2 (Genbank accession AF047976) was included from the end of the N-terminal domain from amino acid position 335. Conserved amino acids among Campanula species are presented in bold text. Consensus among Arabidopsis and Campanula are marked with asterisk. The alignment was produced in T-Coffee (Notredame et al, 2000).

FIG. 3: Expression of ethylene signaling pathway genes in Campanula during flower development. Ggene expression of ERS2, CTRL EIL1 and EIL2 in response to physiological development of flowers in Campanula portenschlagiana Blue GET MEE®, C. formanekiana Blue MARY MEE® and C. medium Sweet MEE®. The developmental stages from bud to flower were; bud (B), flower the day before opening (0), 1-day old flower (1), 2-days old flower (2), and 4-days old flower (4).

FIG. 4: Protein alignment using fragments of putative CTR1 proteins identified in Campanula. As a reference Arabidopsis CTR1 aa 424-663 is included (Genbank accession NP850760). Consensus among Arabidopsis and Campanula are marked with asterisk. The alignment was produced in T-Coffee (Notredame et al, 2000).

FIG. 5: Alignment of EIL2 orthologous in Campanula. 65 bp fragment of EIL2 identified in gDNA of C. formanekiana Blue MARY MEE® and C. medium Blue GET MEE® comprising a 7 bp deletion in C. medium (marked bold). The alignment was produced in T-Coffee (Notredame et al, 2000).

FIG. 6. Expression of putative ethylene signalling pathway genes in Campanula in response to transient ethylene exposure. Gene expression of ERS2, CTR1, EIL1 and EIL2 in response to ethylene concentrations of 0.025 μL·L⁻¹ and 0.05 μL·L⁻¹ of young (1-day old) flowers in Campanula portenschlagiana Blue GET MEE® (CpB), C. formanekiana Blue MARY MEE® (Cf) and C. medium Sweet MEE® (Cm) for 24 h.

FIG. 7. Phylogenetic alignment of EIL orthologous. Overview of conserved regions of different EIL orthologous:

Sequences of approximately 200 aa corresponding to the deduced fragments from the cloned putative ethylene perception genes from Campanula were aligned by ClustalW2 (Larkin et al, 2007). Specific domains are indicated by black boxes. The mutated EIL2 sequence from Campanula medium is highlighted in yellow. The aligned sequences were C. portenschlagiana Blue GET MEE® EIL1 (CpDEIL1), C. portenschlagiana Blue GET MEE® EIL2 (CpDEIL2), Campanula formanekiana Blue MARY MEE® EIL1 (CfEIL1), C. formanekiana Blue MARY MEE® EIL2 (CfEIL2), Campanula medium Sweet MEE® EIL1 (CmEIL1), C. medium Sweet MEE® EIL2 (CmEIL2), Malus×domestica EIL1 (MdEIL1-ADE41153.1), Malus×domestica EIL2 (MdEIL2-ADE41154.1), Malus×domestica EIL3 (MdEIL3-ADE41155.1), Paeonia suffruticosa EIL1 (PsEIL1-AFI61907.1), Paeonia suffruticosa EIL2 (PsEIL2-AFI61908.1), Paeonia suffruticosa EIL3 (PsEIL3-AFI61909.1), Paeonia lactifolia EIN3 (PiEIL3-AFU90136.4), Dianthus caryophyllus EIL1 (DcEIL1-AAF69017.1), Dianthus caryophyllus EIL2 (DcEIL2-AAV68140.1), Dianthus caryophyllus EIL3 (DcEIL3-AAV68141.1), Dianthus caryophyllus EIL4 (DcEIL4-AAV68142.1), Vigna radiata EIL1 (VrEIL1-AAL76272.1), Vigna radiata EIL2 (VrEIL2-AAL76271.1), Phalaenopsis equestris EIN3 (PeEIN3-CAC87091.1), Oncidium hybrid EIL1 (OhEIL1-AEK84143.1), Oncidium hybrid EIL2 (OhEIL2-AEK84144.1), Zea mays EIL1 (ZmEIL1-INP_001152035.1), Musa acuminata EIL1 partial sequence (MaEIL1p-ABG89100.1), Musa acuminata EIL2 partial sequence (MaEIL2p-ABG89101.1), Musa acuminata EIL3 partial sequence (MaEIL3p-ABG89102.1), Musa acuminata EIL4 partial sequence (MaEIL4-ABG89103.1), Arabidopsis thaliana EIN3 on chromosome 3 (AtEIN3.3-024606.1), Arabidopsis thaliana EIN3 on chromosome 5 (AtEIN3.5-NP_201315.1), Arabidopsis thaliana EIL1 (AtEIL1-Q9SLH0.1), Arabidopsis thaliana EIL2 (AtEIL2-O23115.1), Arabidopsis thaliana EIL3 (AtEIL3-NP_177514.1) and Arabidopsis thaliana EIL4 (AtEIL4-AED91496.1).

The alignment shows several EIN/EIL encoded polypeptides from different species. It is observed that 4 highly conserved regions exist, a basic domain region I (BD I), a basic domain region II (BD II), a proline rich region (PR) and a basic domain region III (BD III).

FIG. 8: polypeptide sequence of Campanula medium, cultivars.

Alignment of the translated positive strand of the genomic EIL2 DNA sequences from 8 different Campanula medium cultivars. The alignment shown is zoomed in to the area around the seven base deletion in Campanula medium (corresponding to the highlighted SALI region). The remnant of the SALM motif is highlighted. Unanimous consensus is shown for each amino acids with * in the bottom line. For bases where no unanimous consensus exists, the minority base pair is underlined.

FIG. 9: polypeptide sequence of Campanula medium, cultivars.

Alignment (5′ to 3′) of genomic EIL2 DNA sequences from eight Campanula medium cultivars from a breeding program and two unrelated Campanula medium species. The alignment shown is zoomed in to the area around the seven base deletion in Campanula medium. The bases around the mutation are highlighted. Unanimous consensus is shown for each base with * in the bottom line. For bases where no unanimous consensus exists, the minority base pair is underlined.

FIG. 10: Phylogenetic tree of EIL orthologous.

Deduced proteins of the cloned putative EIL fragments cloned from Campanula were compared by generation of phylogenetic trees by ClustalW2 (http://www.ebi.ac.uk/Tools/msa/clustalw2/).

### DETAILED DESCRIPTION OF THE INVENTION

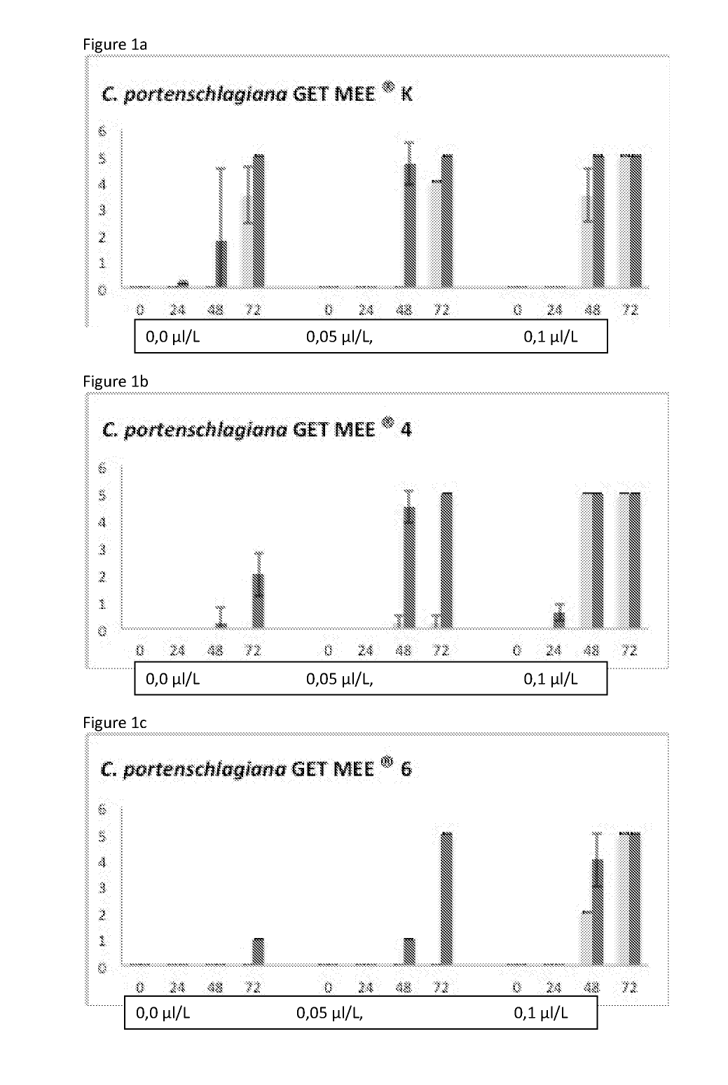

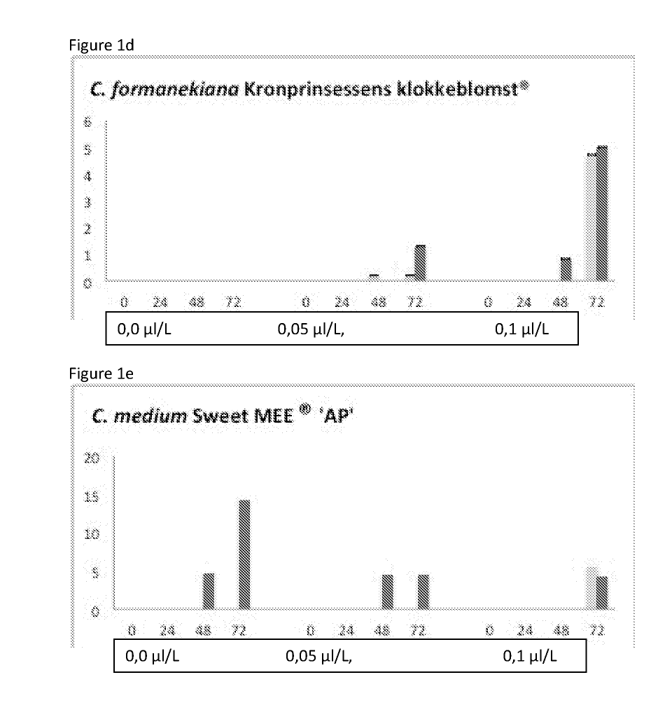

The present invention is based on the finding that cultivars and lines of Campanula medium developed through classical breeding techniques was found to be largely insensitive towards exposure to exogenous ethylene. Thus, it was found that Campanula medium MEE® has reduced sensitivity towards exposure to ethylene, cf. FIG. 1. The nucleic acid sequence relating to this effect has been identified in Campanula medium in the EIL 2 gene. The identification and sequencing of the nucleic acid sequence of Campanula medium provide novel polypeptides. Thus, the present invention provides novel isolated polypeptides and nucleic acid sequences, SEQ ID NO: 3, 4, 11, 12, 13, 14, 15, 16, 17, and 18, which play a role in reducing the ethylene sensitivity in plants.

A change in the ethylene-signaling gene Ethylene Insensitive Like 2 (EIL2) was identified and characterized in Campanula medium, Example 1.

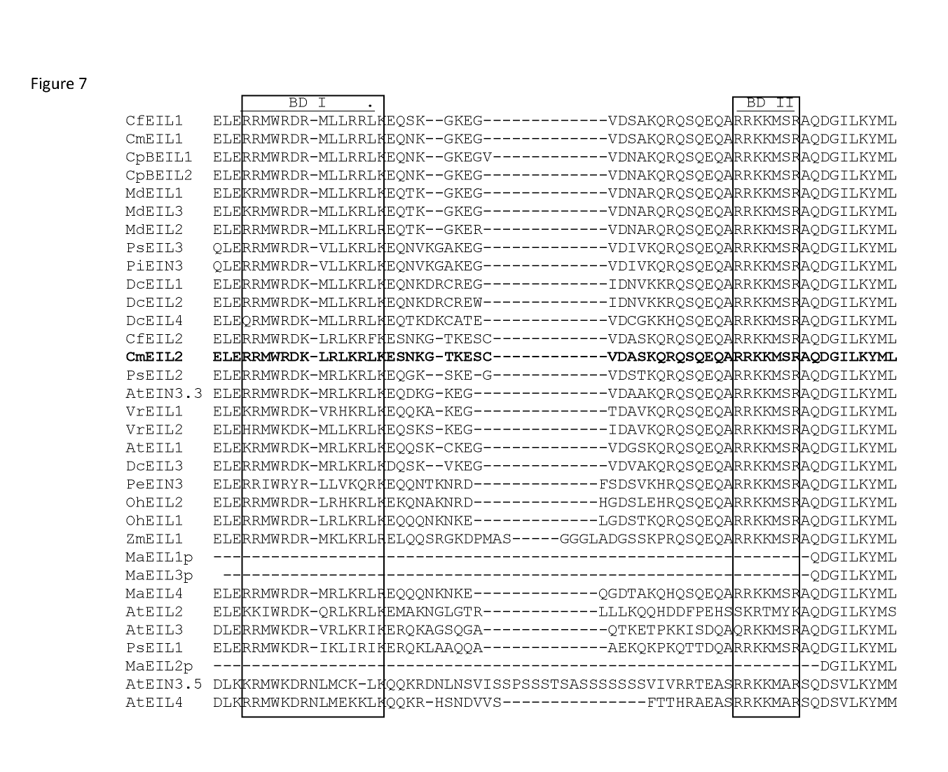

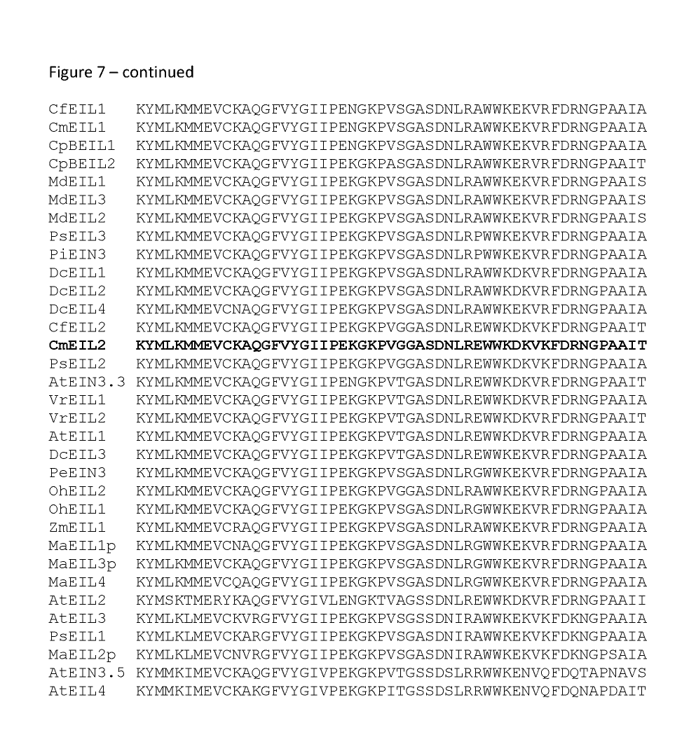

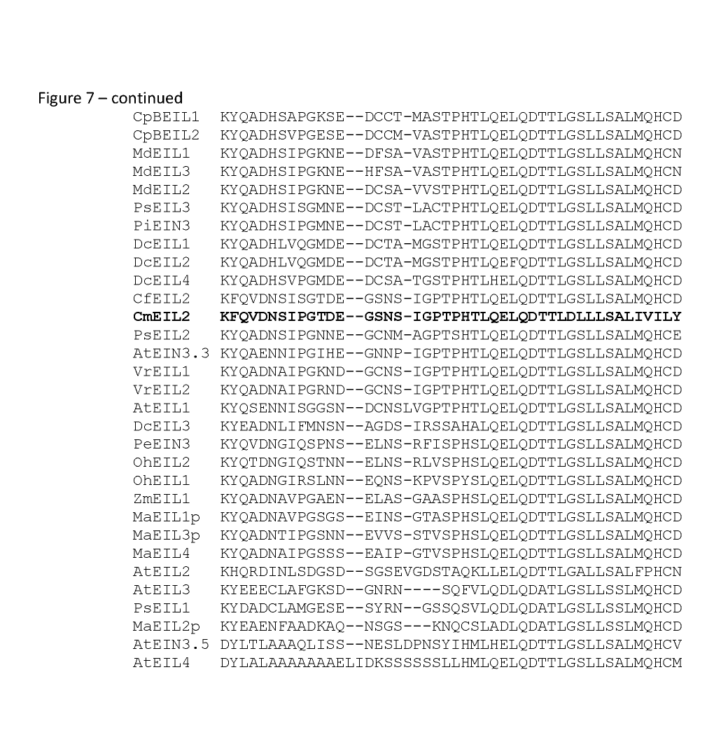

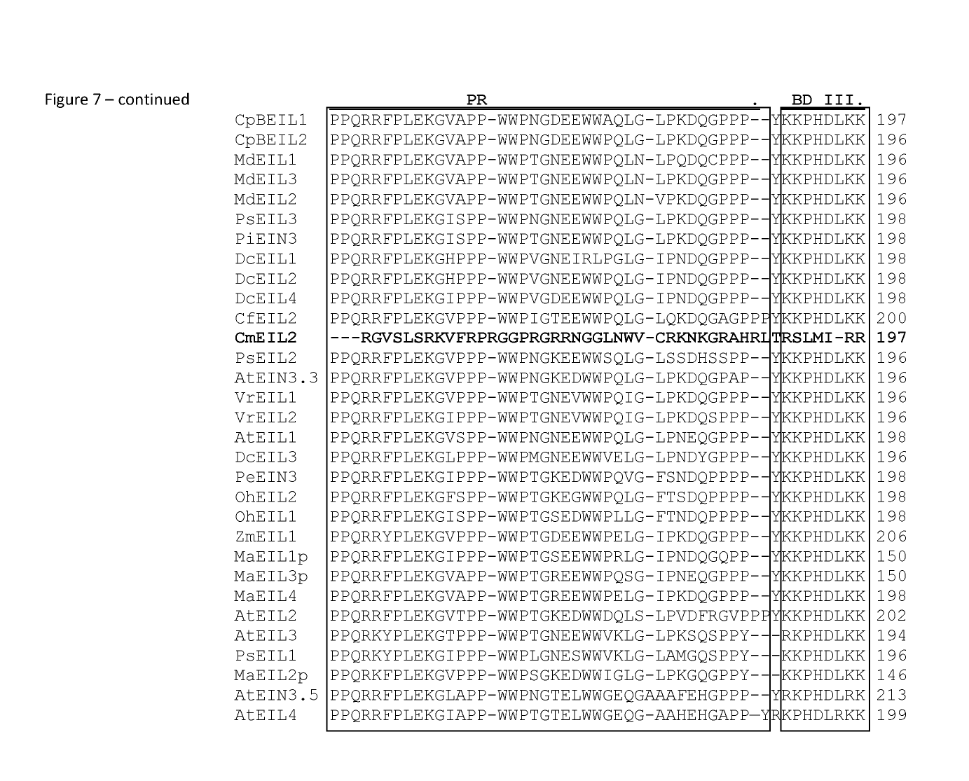

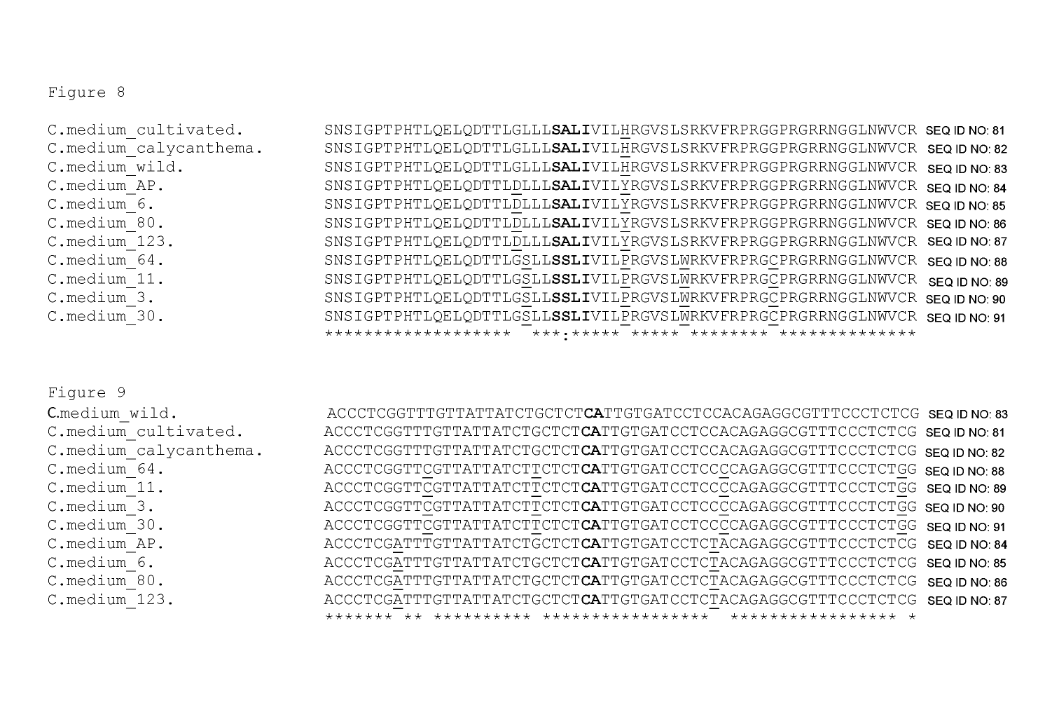

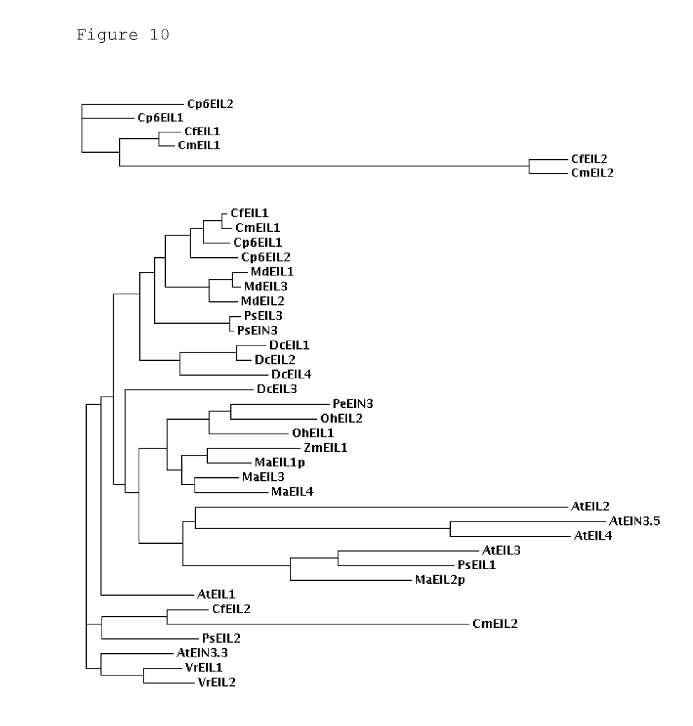

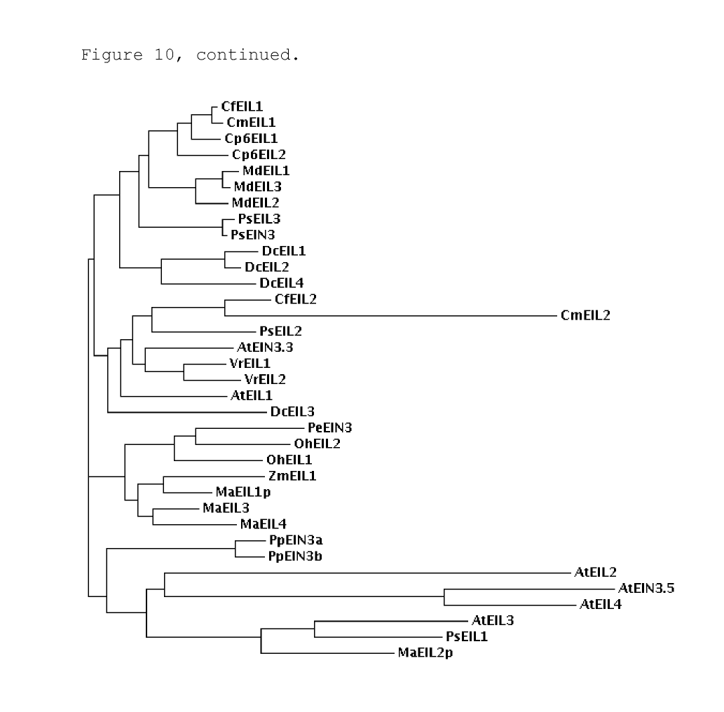

Alignment of the encoded EIL2 protein sequences from Campanula with EIL sequences from other plants showed the presence of two conserved EIL domains consisting of two basic amino acids domain regions (BD I and BD II). Typically, encoded EIN/EIL protein sequences of other plants comprise also a proline rich region (PR) and a third basic domain region (BD III), cf. FIG. 7. High homology was found within EIL from Campanula species except for CmEIL2.

It was further found that the encoded EIL2 polypeptide sequences from Campanula medium MEE® comprises an upstream part and a downstream part divided by a polypeptide motif comprising the amino acid sequence, SSLI.

This encoded protein sequence, SEQ ID NO: 3, exhibits high homology to EIN/EIL polypeptide sequences in other plants in the upstream part of the polypeptide. The downstream part of the polypeptide sequence is highly different to other plants and caused by a frame shift of 7 nucleotides deletion in the nucleic acid sequence of Campanula medium EIL2 polypeptide (CmEIL2). In support of a deletion in the CmEIL2 reading frame is the fact that the frame shift occurs in the conserved SALM polypeptide motif present in most EILs and that omission of the deletion from the CmEIL2 reading frame results in an encoded EIL2 protein sequences that perfectly align with other EIL2 proteins, cf. FIG. 7. Without being bound by theory it appears that a reduced sensitivity towards ethylene can be obtained, if the proline rich region of the encoded EIL2 nucleic acid sequence is destroyed, removed or altered.

The upstream part of Campanula medium may also be modified with one or more amino acids.

EIL2 sequences of the present invention include the disclosed sequences, splice variants, allelic variants, synonymous sequences, and homologous or orthologous variants thereof. Thus, for example, embodiments of the present invention also include genomic and cDNA sequences from the EIL2 gene.

Various nucleic acid constructs in which EIL2 sequences, either complete or parts thereof, may be operable joined to exogenous sequences to form cloning vectors, expression vectors, fusion vectors, transgenic constructs, and the like are also contemplated. In another embodiment of the invention, modified plants having decreased levels of expression of EIN/EIL are provided. Loss of the native EIN/EIL function of the plants was found to result in a decreased sensitivity to ethylene.

Nucleic Acids

The polynucleotides encoding EIL2 polypeptide of the present invention include the nucleotide sequences of SEQ ID NO: 4, 15, 16, 17, and 18. Nucleic acid sequences complementary to these sequences are also encompassed within the present invention.

As used herein, the terms “polynucleotides”, “nucleotides”, and “nucleic acid sequences” refer to DNA, RNA and cDNA sequences. A complementary sequence may include an antisense nucleotide.

When the sequence is RNA, the deoxyribonucleotides A, G, C, and T of SEQ ID NO: 4, 15, 16, 17, and 18 are replaced by ribonucleotides A, G, C, and U, respectively. Also included in the invention are fragments (“probes”) of the above-described nucleic acid sequences that are at least 15 bases in length, which is sufficient to permit the probe to selectively hybridize to DNA that encodes the protein of SEQ ID NO: 3, 11, 12, 13 and 14, or variants thereof.

The term “isolated” as used herein includes polynucleotides or polypeptides substantially free of other nucleic acids, proteins, lipids, carbohydrates or other materials with which it is naturally associated. It is common knowledge of the skilled person to purify such polynucleotides or polypeptides.

The polynucleotide sequences of the invention include DNA, cDNA and RNA sequences which encode an EIL2 polypeptide according to the invention. It is understood that polynucleotides encoding all or varying parts of an EIL2 polypeptide are included, as long as an reduced sensitivity towards exposure to ethylene of the plants can be obtained. Such polynucleotides include naturally occurring, synthetic, and intentionally manipulated polynucleotides as well as splice variants. For example, portions of the mRNA sequence may be altered due to alternate RNA splicing patterns or the use of alternate promoters for RNA transcription.

Moreover, EIL2 polynucleotides include polynucleotides having alterations in the nucleic acid sequence which still encode a polypeptide having the ability to modify the response to ethylene. Alterations in EIL2 nucleic acids include but are not limited to intragenic mutations (e. g., point mutation, nonsense (stop), antisense, splice site and frameshift) and heterozygous or homozygous deletions. Detection of such alterations can be done by standard methods known to those of skill in the art including sequence analysis, Southern blot analysis, PCR based analyses (e.g., multiplex PCR, sequence tagged sites (STSs)) and in situ hybridization. Embodiments of the invention also include anti-sense polynucleotide sequences.

As used herein, the term “polymerase chain reaction” (“PCR”) refers to the method of K. B. Mullis U.S. Pat. Nos. 4,683,195 and 4,683,202, which describes a method for increasing the concentration of a segment of a template sequence in a mixture of genomic DNA without cloning or purification. This process for amplifying the template sequence consists of introducing a large excess of two oligonucleotide primers to the DNA mixture containing the desired template sequence, followed by a precise sequence of thermal cycling in the presence of a DNA polymerase. The two primers are complementary to their respective strands of the double stranded template DNA sequence. To effect amplification, the mixture is denatured and the primers then annealed to their complementary sequences within the template molecule. Following annealing, the primers are extended with a polymerase so as to form a new pair of complementary strands. The steps of denaturation, primer annealing and polymerase extension can be repeated many times (i. e., denaturation, annealing and extension constitute one “cycle”; there can be numerous “cycles”) to obtain a high concentration of an amplified segment of the desired template sequence. The length of the amplified segment of the desired template sequence is determined by the relative positions of the primers with respect to each other, and therefore, this length is a controllable parameter. With PCR, it is possible to amplify a single copy of a specific template sequence in genomic DNA to a level detectable by several different methodologies (e. g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

“Antisense” nucleic acids are DNA or RNA molecules that are complementary to at least a portion of a specific mRNA molecule (Weintraub (1990) Scientific American 262: 40). In the cell, the antisense nucleic acids hybridize to the corresponding mRNA, forming a double-stranded molecule. This interferes with the translation of the mRNA since the cell will not translate a mRNA that is double-stranded. Antisense oligomers of about 15 nucleotides are preferred, since they are easily synthesized and are less likely to cause non-specific interference with translation than larger molecules. The use of antisense methods to inhibit the in vitro translation of genes is well known in the art (Marcus-Sakura (1988) Anal. Biochem. 172: 289). In the present case, plants transformed with constructs containing antisense fragments of the EIL2 gene would display phenotypes with a prolonged shelf life due to delayed ripening, delayed abscission, delayed senescence, and reduced browning of flowers and flower buds.

“Hybridization” refers to the process by which a nucleic acid strand joins with a complementary strand through base pairing. Hybridization reactions can be sensitive and selective so that a particular sequence of interest can be identified even in samples in which it is present at low concentrations. Suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature.

For example, hybridization under high stringency conditions could occur in about 50% formamide at about 37° C. to 42° C. Hybridization could occur under reduced stringency conditions in about 35% to 25% formamide at about 30° C. to 35° C. In particular, hybridization could occur under high stringency conditions at 42° C. in 50% formamide, 5×SSPE, 0.3% SDS, and 200 n/ml sheared and denatured salmon sperm DNA. Hybridization could occur under medium stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

Specifically disclosed herein are genomic and cDNA sequences for EIL2 DNA sequences of the invention can be obtained by several methods. For example, the DNA can be isolated using hybridization or computer-based techniques which are well known in the art. Such techniques include, but are not limited to: 1) hybridization of genomic or cDNA libraries with probes to detect homologous nucleotide sequences; and 2) polymerase chain reaction (PCR) on genomic DNA or cDNA using primers capable of annealing to the DNA sequence of interest;

Screening procedures which rely on nucleic acid hybridization make it possible to isolate any gene sequence from any organism, provided the appropriate probe is available.

Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA clone by the hybridization of the target DNA to that single probe in the mixture which is its complete complement (Wallace, et al., Nucl. Acid Res., 9: 879, 1981). Alternatively, a subtractive library is useful for elimination of non-specific cDNA clones.

Among the standard procedures for isolating cDNA sequences of interest is the formation of plasmid- or phage-carrying cDNA libraries which are derived from reverse transcription of mRNA which is abundant in donor cells that have a high level of genetic expression. When used in combination with polymerase chain reaction technology, even rare expression products can be cloned. In those cases where significant portions of the amino acid sequence of the polypeptide are known, the production of labelled single or double-stranded DNA or RNA probe sequences duplicating a sequence putatively present in the target cDNA may be employed in DNA/DNA hybridization procedures which are carried out on cloned copies of the cDNA which have been denatured into a single-stranded form (Jay, et al., Nucl. Acid Res., 11: 2325, 1983).

Embodiments of the invention also include EIL2 polypeptides, and functional fragments thereof.

The polynucleotides described herein include sequences that are degenerate as a result of the genetic code. There are 20 natural amino acids, most of which are specified by more than one codon. Therefore, all degenerate nucleotide sequences are included in the invention as long as the amino acid sequence of EIL2 polypeptide encoded by such nucleotide sequences retains the plants ability to be insensitive towards exposure to ethylene.

EIL2 Polypeptides

Many modifications of the encoded EIL2 primary amino acid sequence may result in plants having reduced or extinct ethylene responses. Such modifications may be deliberate, as by site-directed mutagenesis, or may be spontaneous. All of the polypeptides produced by these modifications are included herein as long as the biological properties of the encoded EIL2 polypeptide are present. Further, deletion of one or more amino acids can also result in a modification of the structure of the polypeptide without significantly altering its activity.

EIL2 polypeptides include amino acid sequences substantially the same as the sequence set forth in SEQ ID NO: 3, including mutants that result in plants having altered ethylene responsiveness.

For purposes of the present invention, the upstream and/or downstream part are the full sequence of the polypeptide disclosed in SEQ ID NO: 3 may be used as reference sequence to determine the corresponding amino acid sequence of other EIN/EIL polypeptides and for the alignment and calculation of the % identity of these.

Amino acids are denoted either by their commonly known three letter symbols or by the one-letter symbols recommended by the IUPAC-IUB Biochemical Nomenclature Commission. Likewise, nucleotides may be referred to by their commonly accepted single-letter codes.

The term “polypeptide motif” refers to an amino acid sequence, which in the present context comprises at least four amino acids. The relevant motif is SxLx, wherein x denotes any natural amino acid. Thus x may be selected amongst the amino acids, A, C, D, E, F, G, H, I, K, L, M, N, P, Q, R, S, T, V, W, and Y.

The term “substantially the same” refers to amino acid sequences that provide nearly the same amino acid sequence and retain the activity of EIL2 as described herein. Thus, the EIL2 polypeptides of the invention include conservative variations of the polypeptide sequence. The term “conservative variation” as used herein denotes the replacement of an amino acid residue by another, biologically similar residue. Examples of conservative variations include the substitution of one hydrophobic residue such as isoleucine, valine or leucine with another or the substitution of one polar residue for another, such as the substitution of arginine for lysine, glutamic for aspartic acids, or glutamine for asparagine, and the like.

Another aspect of the invention is EIN/EIL polypeptides or fragments thereof, which have at least about 60%, at least about 70%, at least about 80%, at least about 85%, at least about 90%, at least about 95%, or more than about 97% identity to SEQ ID NO: 3. Homology may be determined using any of the methods described herein which align the polypeptides or fragments being compared and determines the extent of amino acid identity or similarity between them, preferably the BLAST method.

Homologous amino acid or nucleotide sequences of the present invention comprise enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Basic Local Alignment Search Tool) (for a review see Altschul, et al., Meth Enzymol. 266: 460, 1996; and Altschul, et al., Nature Genet. 6: 119, 1994).

BLAST is the heuristic search algorithm employed by the programs blastp, blastn, blastx, tblastn, and tblastx using the statistical methods of Karlin and Altschul (available at www.ncbi.nih.gov/BLAST) Altschul, et al., J. Mol. Biol. 215: 403, 1990). The BLAST programs were tailored for sequence similarity searching, for example to identify homologues to a query sequence. The BLAST pages offer several different databases for searching. Some of these databases, such as e. coli, dbEST and month, are subsets of the NCBI (National Center for Biotechnology Information) databases, while others, such as SwissProt, PDB and Kabat are compiled from outside sources. Protein BLAST allows one to input protein sequences and compare these against other protein sequences.

For the present invention the BLAST program is applied under standard algorithms; General parameters; “Short queries”—on/mark, “Expect threshold”—10, “word size”—3, and “max matches in a query range”—0. Scoring parameters; “Matrix”—BLOSUM62, and “Gap costs”—Existence 11 Extension 1. Filter and masking; off/no mark in “low complexity regions”, off/no mark in “mask for lookup table only”, and off/no mark in “mask lower case letters”. If no significant similarity can be found, the “Expect threshold” may be increased to e.g. 100, to search for further sequences. Alternatively, the “word size” may be reduced to 2. If this is not beneficially, the identity of the tested sequence is anticipated as being lower than 10% identity.

For use in alignment purposes the upstream part and downstream part of the polypeptide motif dividing the upstream part and downstream part is located at position 144 to 147 in the corresponding SEQ ID NO: 3. Similarly, this position can be identified in SEQ ID NO: 11, SEQ ID NO: 12, SEQ ID NO: 13, and SEQ ID NO: 14. Correspondingly, the nucleic acid sequence encoding the polypeptide motif can be found in the corresponding SEQ ID NO: 4, which divides the encoded polypeptide in an upstream part and a downstream part. Similarly, such positions can be identified in SEQ ID NO: 15, SEQ ID NO: 16, SEQ ID NO: 17, and SEQ ID NO: 18.

Other computer program methods to determine identity and similarity between the two sequences include but are not limited to the GCG program package (Devereux, et al., Nucl. Acids Res. 12: 387, 1984) and FASTA (Atschul, et al., J Molec. Biol. 215: 403, 1990).

By “percentage identity” is meant % of identical amino acids between the two compared proteins. By “% similarity” is meant the percentage of similar amino acids between the two compared proteins.

One skilled in the art can purify a polypeptide using standard techniques for protein purification to obtain an EIL2 polypeptide that is substantially pure.

As used herein, the term “substantially pure” refers to polypeptides which are substantially free of other proteins, lipids, carbohydrates or other materials with which it is naturally associated. The EIL2 polypeptides can be analyzed by standard SDS-PAGE and/or immunoprecipitation analysis and/or Western blot analysis, for example. The purity of an EIL2 polypeptide can also be determined by aminoterminal amino acid sequence analysis.

Plants with EIL2 Gene Mutations

Another aspect of the invention relates to plants that have been altered to be insensitive to ethylene. Such alterations include transgenic plants with decreased response to ethylene due to transformation with constructs using antisense or co-suppression technology that affect transcription or expression of the EIL2 gene or nucleic acid sequence are also part of the invention.

Such plants exhibit ethylene insensitivity traits such as delayed ripening, delayed flowering, delayed senescence, delayed browning and altered sensitivity to pathogens.

A method is provided for producing a plant or a genetically modified plant characterized as having an altered ethylene-dependent phenotype as compared to a plant which has not been genetically modified (e. g., a wild-type plant). The method includes the steps of contacting plant cells with at least one vector containing at least one nucleic acid sequence encoding an EIN/EIL gene or a mutant, homolog or fragment thereof, wherein the nucleic acid sequence is operably associated with a promoter, to obtain transformed plant cells; producing a number of plants from the transformed plant cells; and thereafter selecting a plant exhibiting a phenotype largely insensitive to ethylene.

In one embodiment, the plant may be modified by classical non-GMO breeding techniques, which are well-recognized by the skilled person. When using these techniques, plants are generally not considered as being transgenic plants, which might be advantageously due to general public concern in relation to transgenic plants.

Another embodiment of the invention includes plants transformed with antisense polynucleotides complementary to the EIL2 gene or fragments thereof wherein production of the antisense polynucleotides results in reduced expression of the EIL2 gene. In an alternative embodiment, reduced expression of EIL2 may be achieved by methods such as co-suppression (Hooper, C. (1991) J. NIH Res. 3: 49-54) by operatively linking a truncated form of an EIL2 gene to a promoter.

The term “genetic modification” as used herein refers to the introduction of one or more heterologous nucleic acid sequences, e. g., an EIL2 or a mutant gene encoding sequence, into one or more plant cells, which can generate whole, sexually competent, viable plants.

The term “genetically modified” as used herein refers to a plant which has been generated through the aforementioned process. Genetically modified plants of the invention are capable of self-pollinating or cross-pollinating with other plants of the same species, or cross-pollinated with closely related species forming interspecific hybrids, so that the foreign gene, carried in the germ line, can be inserted into or bred into agriculturally useful plant varieties.

The term “plant cell” as used herein refers to protoplasts, gamete producing cells, and cells which regenerate into whole plants. Accordingly, a seed comprising multiple plant cells capable of regenerating into a whole plant, is included in the definition of “plant cell”.

As used herein, the term “plant” refers to either a whole plant, a part or an organ of a plant, a plant cell, or a group of plant cells, such as plant tissue, for example. Plantlets are also included within the meaning of “plant”. Plants included in the invention are any plants amenable to transformation techniques, including angiosperms, gymnosperms, monocotyledons and dicotyledons.

Examples of plants include, but are not limited to tomato, Brassica oleracea (e.g., cabbage, broccoli, cauliflower, brussel sprouts), cotton, lettuce, and spinach.

Further, any processed product from the plant or transgenic plant, or a part thereof, such as meal, flour, purified proteins, purified amino acids, and other valued products are also encompassed by the invention.

Other examples of land plants being part of the invention is flowers or flowering plants, which amongst other comprises the families of Asteraceae, Orchidaceae, Fabaceae, Rubiaceae, Poaceae, Lamiaceae, Euphorbiaceae, Melastomataceae, Myrtaceae, Apocynaceae, Cyperaceae, Malvaceae, Araceae, Ericaceae, Gesneriaceae, Apiaceae, Brassicaceae, Piperaceae, Acanthaceae, Rosaceae, Boraginaceae, Urticaceae, Ranunculaceae, Lauraceae, Solanaceae, Campanulaceae, Arecaceae, Annonaceae, Caryophyllaceae, Orobanchaceae, Amaranthaceae, Iridaceae, Aizoaceae, Rutaceae, Phyllanthaceae, Scrophulariaceae, Gentianaceae, Convolvulaceae, Proteaceae, Sapindaceae, Cactaceae, Araliaceae, and Crassulaceae.

Genetically modified plants of the present invention may be produced by contacting a plant cell with a vector including at least one nucleic acid sequence encoding an EIL2 or a variant thereof.

To be effective once introduced into plant cells, the EIL2 nucleic acid sequence is operable associated with a promoter which is effective in the plant cells to cause transcription of EIL2. Additionally, a polyadenylation sequence or transcription control sequence also recognized in plant cells may also be employed. It is preferred that the vector harbouring the nucleic acid sequence to be inserted also contain one or more selectable marker genes so that the transformed cells can be selected from non-transformed cells in culture, as described herein.

Antisense polynucleotide useful for the present invention are complementary to specific regions of a corresponding target mRNA. An antisense polynucleotide can be introduced to a cell by introducing an expressible construct containing a nucleic acid segment that codes for the polynucleotide. Antisense polynucleotides in context of the present invention may include short sequences of nucleic acid known as oligonucleotides, usually 10-50 bases in length, as well as longer sequences of nucleic acid that may exceed the length of the gene sequence itself.

Host Cells and Vectors

DNA sequences encoding EIL2 can be expressed in vitro by DNA transfer into a suitable host cell.

“Host cells” are cells in which a vector can be propagated and its DNA expressed. The term also includes any progeny or graft material, for example, of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

As part of the present invention, the EIL2 polynucleotide sequences may be inserted into a recombinant expression vector.

The terms “recombinant expression vector” or “expression vector” refer to a plasmid, virus or other vehicle known in the art that has been manipulated by insertion or incorporation of the EIL2 genetic sequence. Such expression vectors contain a promoter sequence which facilitates the efficient transcription of the inserted EIL2 sequence. The expression vector typically contains an origin of replication, a promoter, as well as specific genes which allow phenotypic selection of the transformed cells.

Methods which are well known to those skilled in the art can be used to construct expression vectors containing the EIL2 coding sequence and appropriate transcriptional/translational control signals. These methods include in vitro recombinant DNA techniques, synthetic techniques, and in vivo recombination/genetic techniques.

A variety of host-expression vector systems may be utilized to express the EIL2 coding sequence. These include but are not limited to microorganisms such as bacteria transformed with recombinant bacteriophage DNA, plasmid DNA or cosmid DNA expression vectors containing the EIL2 coding sequence; yeast transformed with recombinant yeast expression vectors containing the EIL2 coding sequence; plant cell systems infected with recombinant virus expression vectors (e. g., cauliflower mosaic virus, CaMV; tobacco mosaic virus, TMV) or transformed with recombinant plasmid expression vectors (e. g., Ti plasmid) containing the EIL2 coding sequence; insect cell systems infected with recombinant virus expression vectors (e. g., baculovirus) containing the EIL2 coding sequence; or animal cell systems infected with recombinant virus expression vectors (e. g., retroviruses, adenovirus, vaccinia virus) containing the EIL2 coding sequence, or transformed animal cell systems engineered for stable expression.

Depending on the host/vector system utilized, any of a number of suitable transcription and translation elements, including constitutive and inducible promoters, transcription enhancer elements, transcription terminators, etc. may be used in the expression vector (see e. g., Bitter et al., 1987, Methods in Enzymology 153: 516-544). For example, when cloning in bacterial systems, inducible promoters such as pL of bacteriophage. gamma., plac, ptrp, ptac (ptrp-lac hybrid promoter) and the like may be used. When cloning in mammalian cell systems, promoters derived from the genome of mammalian cells (e. g., metallothionein promoter) or from mammalian viruses (e. g., the retrovirus long terminal repeat; the adenovirus late promoter; the vaccinia virus 7.5K promoter) may be used. Promoters produced by recombinant DNA or synthetic techniques may also be used to provide for transcription of the inserted EIL2 coding sequence.

Isolation and purification of recombinantly expressed polypeptides, or fragments thereof, may be carried out by conventional means including preparative chromatography and immunological separations involving monoclonal or polyclonal antibodies.

Vector (s) employed in the present invention for transformation of a plant cell includes a nucleic acid sequence encoding EIL2, operably linked to a promoter. To commence a transformation process in accordance with the present invention, it is first necessary to construct a suitable vector and properly introduce it into the plant cell. Details of the construction of vectors utilized herein are known to those skilled in the art of plant genetic engineering.

The term “operably linked” refers to functional linkage between a promoter sequence and a nucleic acid sequence regulated by the promoter. The operably linked promoter controls the expression of the nucleic acid sequence.

### Promoters

The expression of structural genes may be driven by a number of promoters. Although the endogenous, or native promoter of a structural gene of interest may be utilized for transcriptional regulation of the gene, preferably, the promoter is a foreign regulatory sequence. For plant expression vectors, suitable viral promoters include the 35S RNA and 195 RNA promoters of CaMV (Brisson, et al., Nature, 310: 511, 1984; Odell, et al., Nature, 313: 810, 1985); the full-length transcript promoter from Figwort Mosaic Virus (FMV) (Gowda, et al., J. Cell Biochem., 13D: 301, 1989) and the coat protein promoter to TMV (Takamatsu, et al., EMBO J. 6: 307, 1987).

Alternatively, plant promoters such as the light-inducible promoter from the small subunit of ribulose bis-phosphate carboxylase (ssRUBISCO) (Coruzzi, et al., EMBO J., 3: 1671, 1984; Broglie, et al., Science, 224: 838, 1984); mannopine synthase promoter (Velten, et al., EMBO J., 3: 2723, 1984) nopaline synthase (NOS) and octopine synthase (OCS) promoters (carried on tumor inducing plasmids of Agrobacterium tumefaciens) or heat shock promoters, e. g., soybean hsp17.5 E or hsp17.3-B (Gurley, et al., Mol. Cell. Biol., 6: 559, 1986; Severin, et al., Plant Mol. Biol., 15: 827, 1990) may be used.

Promoters useful in the invention include both natural constitutive and inducible promoters as well as engineered promoters. The CaMV promoters are examples of constitutive promoters. To be most useful, an inducible promoter should 1) provide low expression in the absence of the inducer; 2) provide high expression in the presence of the inducer; 3) use an induction scheme that does not interfere with the normal physiology of the plant; and 4) have no effect on the expression of other genes. Examples of inducible promoters useful in plants include those induced by chemical means, such as the yeast metallothionein promoter which is activated by copper ions (Mett, et al., Proc. Natl. Acad. Sci., U.S.A., 90: 4567, 1993); Int-1 and Int-2 regulator sequences which are activated by substituted benzenesulfonamides, e. g., herbicide safeners (Hershey, et al., Plant Mol. Biol., 17: 679, 1991); and the GRE regulatory sequences which are induced by glucocorticoids (Schena, et al., Proc. Natl. Acad. Sci., U.S.A, 88: 10421, 1991). Other promoters, both constitutive and inducible will be known to those of skill in the art.

The particular promoter selected should be capable of causing sufficient expression to result in the production of an effective amount of structural gene product to alter an ethylene-dependent phenotype. The promoters used in the vector constructs of the present invention may be modified, if desired, to affect their control characteristics.

Tissue specific promoters may also be utilized in the present invention. An example of a tissue specific promoter is the promoter active in shoot meristems (Atanassova, et al., Plant J., 2: 291, 1992). Other tissue specific promoters useful in transgenic plants, including the cdc2a promoter and cyc07 promoter, will be known to those of skill in the art. (See for example, Ito, et al., Plant Mol. Biol., 24: 863, 1994; Martinez, et al., Proc. Natl. Acad. Sci. USA, 89: 7360, 1992; Medford, et al., Plant Cell, 3: 359, 1991; Terada, et al., Plant Journal, 3: 241, 1993; Wissenbach, et al., Plant Journal, 4: 411, 1993).

Optionally, a selectable marker may be associated with the nucleic acid sequence to be inserted.

As used herein, the term “marker” refers to a gene encoding a trait or a phenotype which permits the selection of, or the screening for, a plant or plant cell containing the marker.

Preferably, the marker gene is an antibiotic resistance gene whereby the appropriate antibiotic can be used to select for transformed cells from among cells that are not transformed. Examples of suitable selectable markers include adenosine deaminase, dihydrofolate reductase, hygromycin-Bphospho-transferase, thymidine kinase, xanthine-guanine phospho-ribosyltransferase and aminoglycoside 3′-O-phospho-transferase II (kanamycin, neomycin and G418 resistance). Other suitable markers will be known to those of skill in the art.

### Methods of Molecular Breeding

The mutation of EIL2 in Campanula medium (Cm) is positioned in a highly conserved region, and it therefore presents a large potential for molecular breeding towards higher tolerance to ethylene in other plant species. The mutation of EIL2 in Cm could be utilized in trans- and cis-genesis approaches, as well as in targeted mutagenesis techniques using Zinc finger nucleases (ZNF), Transcription activator-like effector nucleases (TALENs) (Lusser et al, 2012) or Clustered regularly interspaced palindromic repeats/CRISPR-associated 9 (CRISPR/Cas9) (Belhaj et al, 2013). With these approaches, increased ethylene tolerance may be transferred from Cm to other important climacteric ornamentals e.g. roses, petunia, carnations, or even edible climacteric crops such as broccoli, banana and tomato.

Protocols for transformation of Campanula carpatica have already been established, with a focus of future production of ethylene resistant plants (Sriskandarajah et al, 2004). Transgenic C. carpatica etr1-1 plants showing reduced ethylene sensitivity have successfully been obtained (Sriskandarajah et al, 2007). However, isolation of the mutated EIL2 gene opens up the possibility of alternative strategies.

Methods and compositions to target and cleave genomic DNA by site specific nucleases ZFNs, Meganucleases, CRISPR/Cas9 system and TALENs) have been developed.

The site specific cleavage of genomic loci by ZFNs can be used, for example, to induce targeted mutagenesis, induce targeted deletions of cellular DNA sequences, and facilitate targeted recombination of an exogenous donor DNA polynucleotide within a predetermined genomic locus. See, for example, U.S. Patent Publication No. 20030232410; 20050208489; 20050026157; 20050064474; and 20060188987, and International Patent Publication No. WO 2007/014275, these patent applications are hereby incorporated by reference in their entireties for all purposes. U.S. Patent Publication No. 20080182332 describes use of non-canonical ZFNs for targeted modification of plant genomes and U.S. Patent Publication No. 20090205083 describes ZFN-mediated targeted modification of a plant EPSPs genomic locus. In addition, Moehle et al. (2007) Proc. Natl. Acad. Sci. USA 104(9): 3055-3060 describe using designed ZFNs for targeted gene addition at a specified genomic locus. The skilled person would know how to employ ZFN for use according to the present invention.

Targeted genome engineering or genome editing has emerged as an alternative to classical plant breeding and transgenic (GMO) methods to improve crop plants. Until recently, available tools for introducing site-specific double strand DNA breaks were restricted to ZFNs and TALENs. However, the CRISPR/Cas9 system is another method that allows for targeted cleavage of genomic DNA guided by a customizable small noncoding RNA, resulting in gene modifications by both non-homologous end joining (NHEJ) and homology-directed repair (HDR) mechanisms, cf. Belhaj et al, 2013. Thus, in one embodiment, the CRISPR/Cas9 method may be used to provide new plants, which are less sensitive towards ethylene.

Current methods of targeting typically involve co-transformation of plant tissue with a donor DNA polynucleotide containing at least one transgene and a site specific nuclease (e.g., ZFN) which is designed to bind and cleave a specific genomic locus. The donor DNA polynucleotide is stably inserted within the cleaved genomic locus resulting in targeted gene addition at a specified genomic locus.

EIL2 nucleic acid sequences utilized in the present invention can also be introduced into plant cells using Ti plasmids of Agrobacterium tumefaciens. (For reviews of such techniques see, for example, Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9, and Horsch, et al., Science, 227: 1229, 1985). In addition to plant transformation vectors derived from the Ti plasmids of Agrobacterium, alternative methods may involve, for example, the use of liposomes, electroporation, chemicals that increase free DNA uptake, transformation using viruses or pollen and the use of microprojection.

One of skill in the art will be able to select an appropriate vector for introducing the EIL2 nucleic acid sequence in a relatively intact state. Thus, any vector which will produce a plant carrying the introduced DNA sequence should be sufficient. Even use of a single stranded DNA would be expected to confer the properties of this invention, though at low efficiency. The selection of the vector, or whether to use a vector, is typically guided by the method of transformation selected.

The transformation of plants in accordance with the invention may be carried out in essentially any of the various ways known to those skilled in the art of plant molecular biology. (See, for example, Methods of Enzymology, Vol. 153, 1987, Wu and Grossman, Eds., Academic Press). As used herein, the term “transformation” means alteration of the genotype of a host plant by the introduction of an EIL2 gene, or a mutant form of the EIL2 gene.

For example, an EIL2 nucleic acid sequence can be introduced into a plant cell utilizing Agrobacterium tumefaciens containing the Ti plasmid, as mentioned briefly above. In using an A. tumefaciens culture as a transformation vehicle, it is most advantageous to use a nononcogenic strain of Agrobacterium as the vector carrier so that normal non-oncogenic differentiation of the transformed tissues is possible. It is also preferred that the Agrobacterium harbour a binary Ti plasmid system. Such a binary system comprises 1) a first Ti plasmid having a virulence region essential for the introduction of transfer DNA (T-DNA) into plants, and 2) a chimeric plasmid. The latter contains at least one border region of the T-DNA region of a wildtype Ti plasmid flanking the nucleic acid to be transferred. Binary Ti plasmid systems have been shown effective to transform plant cells (De Framond, Biotechnology, 1: 262, 1983; Hoekema, et al., Nature, 303: 179, 1983). Such a binary system is preferred because it does not require integration into the Ti plasmid of Agrobacterium, which is an older methodology.

Methods involving the use of Agrobacterium in transformation according to the present invention include, but are not limited to: 1) co-cultivation of Agrobacterium with cultured isolated protoplasts; 2) transformation of plant cells or tissues with Agrobacterium; or 3) transformation of seeds, apices or meristems with Agrobacterium.

In addition, gene transfer can be accomplished by in planta transformation by Agrobacterium, as described by Bechtold, et al., (C. R. Acad. Sci. Paris, 316: 1194, 1993). This approach is based on the vacuum infiltration of a suspension of Agrobacterium cells.

One method of introducing EIL2 nucleic acid sequences into plant cells is to infect such plant cells, an explant, a meristem or a seed, with transformed Agrobacterium tumefaciens as described above. Under appropriate conditions known in the art, the transformed plant cells are grown to form shoots, roots, and develop further into plants.

Alternatively, EIL2 nucleic acid sequences can be introduced into a plant cell using mechanical or chemical means. For example, the nucleic acid can be mechanically transferred into the plant cell by microinjection using a micropipette. Alternatively, the nucleic acid may be transferred into the plant cell by using polyethylene glycol which forms a precipitation complex with genetic material that is taken up by the cell.

EIL2 nucleic acid sequences can also be introduced into plant cells by electroporation (Fromm, et al., Proc. Natl. Acad. Sci., U.S.A, 82: 5824, 1985). In this technique, plant protoplasts are electroporated in the presence of vectors or nucleic acids containing the relevant nucleic acid sequences. Electrical impulses of high field strength reversibly permeabilize membranes allowing the introduction of nucleic acids. Electroporated plant protoplasts reform the cell wall, divide and form a plant callus. Selection of the transformed plant cells with the transformed gene can be accomplished using phenotypic markers as described herein.

Another method for introducing EIL2 nucleic acid into a plant cell is high velocity ballistic penetration by small particles with the nucleic acid to be introduced contained either within the matrix of such particles, or on the surface thereof (Klein, et al., Nature 327: 70, 1987). Bombardment transformation methods are also described in Sanford, et al. (Techniques 3: 316, 1991) and Klein, et al. (Bio/Techniques 10: 286, 1992). Although, typically only a single introduction of a new nucleic acid sequence is required, this method particularly provides for multiple introductions. In this technique a particle, or microprojectile, coated with DNA is shot through the physical barriers of the cell. Particle bombardment can be used to introduce DNA into any target tissue that is penetrable by DNA coated particles, but for stable transformation, it is imperative that regenerable cells be used. Typically, the particles are made of gold or tungsten. The particles are coated with DNA using either CaCl₂ or ethanol precipitation methods which are commonly known in the art. DNA coated particles are shot out of a particle gun. A suitable particle gun can be purchased from Bio-Rad Laboratories (Hercules, Calif., USA). Particle penetration is controlled by varying parameters such as the intensity of the explosive burst, the size of the particles, or the distance particles must travel to reach the target tissue. The DNA used for coating the particles may comprise an expression cassette suitable for driving the expression of the gene of interest that will comprise a promoter operably linked to the gene of interest.

After cloning, the recombinant plasmid again may be cloned and further modified by introduction of the desired nucleic acid sequence. The modified viral portion of the recombinant plasmid is then excised from the parent bacterial plasmid, and used to inoculate the plant cells or plants.

As used herein, the term “contacting” refers to any means of introducing EIL2 into the plant cell, including chemical and physical means as described above. Preferably, contacting refers to introducing the nucleic acid or vector into plant cells (including an explant, a meristem or a seed), via Agrobacterium tumefaciens transformed with the EIL2 nucleic acid as described above.

Normally, a plant cell is regenerated to obtain a whole plant from the transformation process. The term “producing” or “regeneration” as used herein means producing a whole plant from a plant cell, a group of plant cells, a plant part (including seeds), or a plant piece (e. g., from a protoplast, callus, or tissue part). Regeneration from protoplasts varies from species to species of plants, but generally a suspension of protoplasts is first made. In certain species, embryo formation can then be induced from the protoplast suspension, to the stage of ripening and germination as natural embryos. The culture media will generally contain various amino acids and hormones, necessary for growth and regeneration. Examples of hormones utilized include auxins and cytokinins. It is sometimes advantageous to add glutamic acid and proline to the medium. Efficient regeneration will depend on the medium, on the genotype, and on the history of the culture. If these variables are controlled, regeneration is reproducible.

Regeneration also occurs from plant callus, explants, organs or parts. Transformation can be performed in the context of organ or plant part regeneration. (see Methods in Enzymology, Vol. 118 and Klee, et al., Annual Review of Plant Physiology, 38: 467, 1987). Utilizing the leaf disctransformation-regeneration method of Horsch, et al., Science, 227: 1229, 1985, discs are cultured on selective media, followed by shoot formation in about 2-4 weeks.

In vegetatively propagated crops, the mature plant or mature transgenic plants are propagated by utilizing cuttings or tissue culture techniques to produce multiple identical plants. Selection of desirable varieties is made and new varieties are obtained and propagated vegetatively for commercial use.

In seed propagated crops, the mature plant or transgenic plants can be self crossed to produce a homozygous inbred plant. The resulting inbred plant produces seed containing the newly introduced foreign gene(s) or nucleic acid sequence. These seeds can be grown to produce plants that exhibit an altered response to ethylene.

Parts obtained from regenerated plants, such as flowers, seeds, leaves, branches, roots, fruit, and the like are included in the invention, provided that these parts comprise cells that have been transformed as described. Progeny and variants, and mutants of the regenerated plants are also included within the scope of the invention, provided that these parts comprise the introduced nucleic acid sequences.

Plants exhibiting an altered ethylene-dependent phenotype as compared with wild-type plants can be selected by visual observation. For example, since ethylene affects a wide group of plant characteristics, such as leaf curling, flowering, fruit ripening and senescence, one of skill in the art would be able to design other types of visual screens for altered responses to ethylene, depending on the plant species being tested. Leaf curling is advantageous, since seedlings with reduced leaf curling can be detected early. Types of screens that could be designed include, for example, screens for delayed leaf or flower senescence or delayed fruit ripening in response to ethylene application.

In some embodiments of the invention, ethylene gas may be used to analyze ethylene response phenotypes. Additionally, molecules which are precursors to ethylene may be used.

Plants having reduced EIL2 activity and thus a reduced sensitivity to ethylene could be useful for the floral industry. Since ethylene may be involved in floral senescence, these modified plants may have a longer flower longevity. Further, it may be useful to use the present invention to create vegetative crops that do not bolt or flower easily. Yellowing of leafy vegetables like broccoli, lettuce and salad be reduced by this mutation. Because ethylene has been implicated in senescence, potted plants made according to the present invention may last longer than control plants due to reduced leaf senescence.

One of skill in the art would appreciate that different plants or different agricultural crops may benefit from different types of promoter/EIL2 modifications. For example, in some plants it may be desirable to be able to decrease the sensitivity constitutively using, for example, a CaMV35S promoter linked to the modified EIL2 gene. Other plants may benefit from decreasing the sensitivity to ethylene only at fruit ripening, for example, by linking the modified EIL2 gene to a fruit ripening-specific promoter. It may be useful to create plants that have modified ethylene sensitivity in the vegetative parts of the plant. In another example, it may be useful to prepare plants having a modified EIL2 gene so that the ethylene sensitivity is decreased only at high temperatures. Such a scenario may be important in postharvest storage and transportation of fruit, for example. It may be useful to link the gene to a promoter such that the ethylene insensitivity characteristic is brought about only when the plant is stored in the darkness by operably linking a darkness-inducible promoter to the modified EIL2 gene. This may be particularly useful for managing long term storage of certain crops between the time of harvest and the time of display for sale.

The ability to control the sensitivity of plants to ethylene could thus significantly improve the quality and longevity of many plants, such as ornamental plants, flowers, and climacteric crops.

It is to be understood that this invention is not limited to specific compositions or process steps, as such may vary. It must be noted that, as used in this specification and the appended claims, the singular form “a”, “an” and “the” include plural referents unless the context clearly dictates otherwise. Unless defined otherwise, all technical and scientific terms used herein have the same meaning as commonly understood by one of ordinary skill in the art to which this invention is related.

### REFERENCE TO A SEQUENCE LISTING

This application contains a Sequence Listing in computer readable form, which is incorporated herein by reference.

SEQ ID NO: 1: polypeptide sequence of Campanula formanekiana EIL1.

SEQ ID NO: 2: polypeptide sequence of Campanula formanekiana EIL2, including the upstream part, the polypeptide motif (SALM) and the downstream part.

SEQ ID NO: 3: polypeptide sequence of Campanula medium EIL2.

SEQ ID NO: 4: nucleic acid sequence encoding SEQ ID NO: 3.

SEQ ID NO: 5: polypeptide sequence of Campanula medium EIL1

SEQ ID NO: 6: polypeptide sequence of Campanula portenschlagiana EIL1.

SEQ ID NO: 7: polypeptide sequence of Campanula portenschlagiana EIL2.

SEQ ID NO: 8: polypeptide sequence of Arabidopsis thaliana EIL1 including the upstream part, the polypeptide motif (SALM) and the downstream part.

SEQ ID NO: 9: upstream part of SEQ ID NO: 3, including SALI polypeptide motif.

SEQ ID NO: 10: Downstream part of Arabidopsis thaliana, AtEIL1-Q9SLH0.1.

SEQ ID NO: 11: Campanula medium EIL2, cultivar 64, including the upstream part, the polypeptide motif (SSLI) and the downstream part.

SEQ ID NO: 12: Campanula medium EIL2, cultivar 11, including the upstream part, the polypeptide motif (SSLI) and the downstream part.

SEQ ID NO: 13: Campanula medium EIL2, cultivar 3, including the upstream part, the polypeptide motif (SSLI) and the downstream part.

SEQ ID NO: 14: Campanula medium EIL2, cultivar 30, including the upstream part, the polypeptide motif (SSLI) and the downstream part.

SEQ ID NO: 15: nucleic acid sequence encoding SEQ ID NO: 11.

SEQ ID NO: 16: nucleic acid sequence encoding SEQ ID NO: 12.

SEQ ID NO: 17: nucleic acid sequence encoding SEQ ID NO: 13.

SEQ ID NO: 18: nucleic acid sequence encoding SEQ ID NO: 14.

SEQ ID NO: 19: Upstream part of Arabidopsis thaliana, AtEIL1-Q9SLH0.1.

### EXAMPLES

### Example 1

Plant Material and Growth Conditions

Campanula plants were received from the nursery PKM (Odense, Denmark) in a developmental stage with only young buds. The plants were transferred directly to a greenhouse with 18° C. day/15° C. night and a 16 h photoperiod of natural light for further development. A list of the investigated Campanula species is presented in Table 1.

TABLE 1
Overview of plants used in the experiment

| Scientific name | Cultivar | Abbreviation |
|---|---|---|
| Campanula portenschlagiana | White GET MEE ® | CpW |
| Schultes. | | |
| Campanula portenschlagiana | Dark GET MEE ® | CpD |
| Schultes. | | |
| Campanula portenschlagiana | Blue GET MEE ® | CpB |
| Schultes. | | |
| Campanula formanekiana | Blue MARY MEE ® | Cf |
| Degen & Doefler. | | |
| Campanula medium L. | Sweet MEE ® | Cm |

Identification of Putative ERS2, CTR1 and EIL Genes

Based on the NCBI gene bank (http://ncbi.nlm.nih.gov/) sequence from Campanula carpatica (AF413669) primers were designed to amplify partial fragments of ERS2. ERS2 primers were designed to be intron spanning. For amplification of partial fragments of CTR1, sequences from Musa accuminata, Solanum lycopersicon, Arabidopsis thaliana and Rosa hybrid (JF430422, AF096250, NM_180429, and AY032953) were used to design degenerated primers in conserved areas among the species. Similarly, degenerated primers specific for fragments of EIL genes were designed based on sequences of Malus×domestica and Solanum lycopersicon (GU732486 and NM_001247617). Primer sequences are available in Table 2.

TABLE 2
primers used for identification of putative Campanula
ethylene perception genes

| Gene | Forward primer (5′-3′) | Reverse primer (5′-3′) |
|---|---|---|
| ERS2 | TGTGGAAGTTGTTGCTGACC | AGCTAGGAAATCATTACGAGCA |
| CTRI | CGAATTGCCAARGGMTGTAA | ATTCATYCCGTTTGCCACATC |
| EIL1 | WGAGCTMGAGAGGAGGATGTG | GCCTTCTTCAGATCATGAGGC |
| EIL2 | GGGGACGAAGGAATCATGTGTT | CGCCCCTTGATCCTTCTGCAAA |
| ACTIN | GCAGGACGTGATCTGACTGA | CGCCCCTTGATCCTTCTGCAAA |

Genomic DNA from CpB, CpD, CpW, Cf, and Cm was isolated from flowers with DNeasy plant mini kit (Qiagen, Hilden, Germany) using 300 mg of plant material following manufacturer's recommendations. PCR was performed with 100-250 ng gDNA, 2% (v/v) DMSO, using LaTaq (Clontech Lab Inc, Takara, Mountain View, Calif., USA) as polymerase according to manufacturer's recommendations in total volumes of 25 μl. DNA amplification was performed in a thermal cycler (MyCycler, Biorad, Hercules, Calif., USA) using the following program; 4 min 94° C., 33-35 cycles of [30 s 94° C., 1 min 60° C., 1 min 72° C.] and a final 7 min elongation step at 72° C. Cloning of the PCR-products was done using TOPO TA Cloning® kit (Life Technologies Corp, Invitrogen, Carlsbad, Calif., USA) following manufacturer's recommendations. Plasmids were purified by QIAprep Spin Miniprep kit (Qiagen, Hilden, Germany) and sequenced using Eurofins MWG Operon (Ebersberg, Germany).

RNA Extraction, cDNA Synthesis and Expression Analysis

RNA from CpB, CpD, CpW, Cf, and Cm was isolated from flowers with RNeasy mini kit (Qiagen, Hilden, Germany) using 100 mg of plant material ground in liquid nitrogen. The RNA isolation procedure was performed with some changes to manufacturer's protocol: RLT buffer was used together with β-mercaptoethanol 0.01% (v/v) to lyse the cells. This step included a 1 min incubation at 56° C. RNA yield and purity was measured on a Nanodrop (Thermo Fisher Scientific Inc. Wilmington, Del., USA). Purified RNA was stored at −80° C. or DNAse treated with Amplification Grade DNase I (Invitrogen, Carlsbad, Calif., USA) using 800 ng RNA template. cDNA synthesis was done using iScript cDNA Synthesis kit as recommended (Biorad, Hercules, Calif., USA). Expression analysis were performed in a MyCycler (Biorad, Hercules, Calif., USA) in a 25 μL reaction mixture containing five times diluted cDNA and using ExTaq as polymerase (Clontech Lab Inc, Takara, Mountain View, Calif., USA). Primers and gene specific reaction settings used in expression analysis are presented in Table 4. The following PCR program was used; 4 min 94° C., 25-32 cycles of [30 s 94° C., 1 min 55° C., 1 min 72° C.] and a final step of 7 min at 72° C. All RT-PCR analyses were performed in two biological replicates starting from the harvest of flowers.

TABLE 4
RT-PCR primers for expression analyses

| Gene | Forward primer (5′-3′) | Reverse primer (5′-3′) |
|---|---|---|
| ERS2 | TGTGGAAGTTGTTGCTGACC | AGCTAGGAAATCATTACGAGCA |
| CTR1 | GTTCGATTTGGGATTGACAGG | CCAATGACCTGAAATCAATGGTTG |
| CpBEIL1 | GCGAGGACTGCTGTACGATG | GCCTTCTTCAGATCATGAGGC |
| CpBEIL2 | GTGAGGACTGCTGTATGGTT | GCCTTCTTCAGATCATGAGGC |
| CfEIL1 | ACCTCACACATTGCAGGAA | GCCTTCTTCAGATCATGAGGC |
| CfEIL2 | CAGAGGCGTTTCCCTCT | GCCTTCTTCAGATCATGAGGC |
| CmEIL1 | CAGCTTTGATGCAGCATTGTG | GCCTTCTTCAGATCATGAGGC |
| CmEIL2 | CTGCTCTCATTGTGATCCTCTA | GCCTTCTTCAGATCATGAGGC |
| ACTIN | GCAGGACGTGATCTGACTGA | GGGAACATRGTTGAWCCACCAC |

Ethylene Exposure Experiments

To monitor flower development individual buds were labelled one day before flower opening. This stage was termed day 0. In the following days newly opened flowers (day 1) and 4 days old flowers (day 4) were identified, tagged and used in subsequent experiments. This allowed two morphologically different stages to be monitored simultaneously throughout the ethylene exposure experiment. Ethylene exposure experiments were conducted in a climate chamber in glass tanks with postharvest growth conditions; 20° C. day/18° C. night, 16 h photoperiod at 10-12 μmol m⁻² s⁻¹ provided by cool-white fluorescent tubes (Philips Master TL-D-36W/830, Amsterdam, The Netherlands). Each glass tank had a volume of 128 L and contained three plants. On each plant, five day 1 and five day 4 flowers were marked on plants of all species except C. medium, which did not develop five flowers at the same developmental stage at the same time. As a result, each glass tank contained three plants with a total of 15 labelled flowers for each developmental stage (day 1 and day 4). Two ethylene concentrations of 0.05 μL/L or 0.1 μL/L ethylene, respectively, and a control (0 μL/L) were obtained by injection of gaseous ethylene (Mikro lab gas, Mikrolab Aarhus A/S, Hoejbjerg, Denmark) into the sealed glass tanks. Every 24 h the glass tanks were opened to monitor the number of senesced flowers and to ventilate the tanks. Following ventilation ethylene was re-injected with the respective ethylene concentrations and the tanks were sealed. Each tank was monitored every 24 h over a period of 72 h. A senescent flower was defined as a flower showing twisted or closed corolla or wilted. Each experiment was performed in two biological replicates.

Gene Expression

Gene expression analyses were done using flowers at the developmental stages: unripe bud, day 0 (one day before flowering), day 1, day 2 and day 4. Plants were subjected to 24 h ethylene exposure experiments conducted with low ethylene concentrations of 0.025 μL/L and 0.05 μL/L ethylene in glass tanks. Each glass tank contained three plants each with five tagged day 1 flowers providing a total of 15 flowers per RNA extraction. Following ethylene injection tanks were sealed for 24 h. As control tanks with no ethylene injections were used. After 24 h, flowers were harvested in liquid nitrogen and stored at −80° C. for RNA extraction. Each experiment was performed in two biological replicates.

Bioinformatics

Sequence identification and analysis were done using CLC (CLC bio, www.cicbio.com), BLAST and T-Coffee (Notredame et al, 2000). The phylogenetic trees were constructed using ClustalW2 (Larkin et al, 2007).

Results

The physiological and molecular responses of three Campanula species to exogenous ethylene were characterised. Key genes in Campanula flower ethylene perception and signalling were identified and expression levels characterized at various stages of flower development and in response to exogenous ethylene. Exploring the differences in ethylene sensitivity on the molecular level, a novel mutation was identified as an explanation for the ethylene insensitivity of C. medium.

Differential Ethylene Sensitivity within Campanula

To understand the natural diversity within the Campanula genus towards ethylene sensitivity, three commercially important species were selected; Campanula portenschlagiana (Cp) characterized by small bell-shaped flowers, Campanula medium (Cm) and Campanula formanekiana (Cf), both representing large bell-flower species (FIG. 1). Within Cp the cultivars Dark GET MEE®, Blue GET MEE® and White GET MEE® were included as representatives of small flowered plant lines. Ethylene exposure tests were conducted for Oh, 24 h, 48 h and 72 h, respectively, with concentrations of 0 μL/L, 0.05 μL/L and 0.1 μL/L ethylene in sealed glass tanks. Before exposure of the plants, two developmental stages of flowers (day 1 and day 4) representing young and old flowers, were marked to ensure comparison of flowers at the same stage of development.

Among the Campanula species investigated, the physiological responses to ethylene exposure were diverse. The small flowered species Cp were ethylene sensitive, flowers of the clones 4 and 6 senesced following exposure to 0.1 μL/L ethylene already after 48 hours exposure. For both lines, older flowers were more sensitive to ethylene than younger flowers. CpW was the most sensitive plant line reacting strongly with senescing flowers in the closed tank, even without added ethylene. Young and old flowers of CpW senesced in response to 72 hours containment in the tanks, regardless of ethylene presence. Control CpW plants in the same climate chamber placed in an open tank did not respond with wilting flowers (data not shown). The large-flower species Cf was sensitive to 0.1 μL/L ethylene after 72 hours exposure responding with senescing flowers; however the difference between young and old flowers sensitivity to ethylene was not profound in this plant species. Finally, the large flowers of Cm were able to tolerate even 0.1 μL/L ethylene during 72 hours without any flowers wilted.

Identification of Putative Ethylene Perception and Signal Transduction Genes

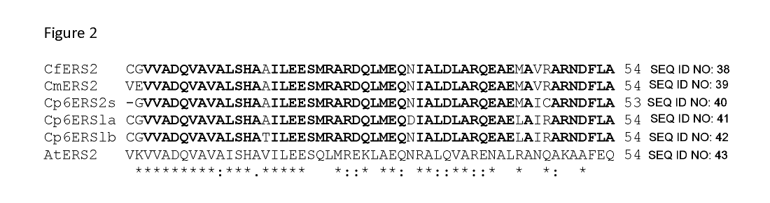

Further studies on the molecular level were conducted to unravel the observed differences in ethylene sensitivity. To clarify whether Campanula perceive and transduce ethylene signals via the same pathway as described in other plants, three key genes were selected for investigation: ERS2, CTR1, and EIL. Primers (Table 2) were designed based on gene homology from other plant species and the sequence identities within Campanula were verified by sequencing. ERS2 specific primers using genomic DNA as a template produced two ERS2 fragments in CpW, CpD and CpB and only one ERS2 gene fragment in both Cf and Cm. Following sequencing and transcriptional analysis it became evident that the two CpERS2 fragments were derived from two similar ERS2 homologs with differences in intron sizes (FIG. 2, only data for CpB is shown). Alignment of the putative protein fragment of Campanula ERS2 showed a higher sequence identity within Cf and Cm than compared to Cp (FIG. 2).

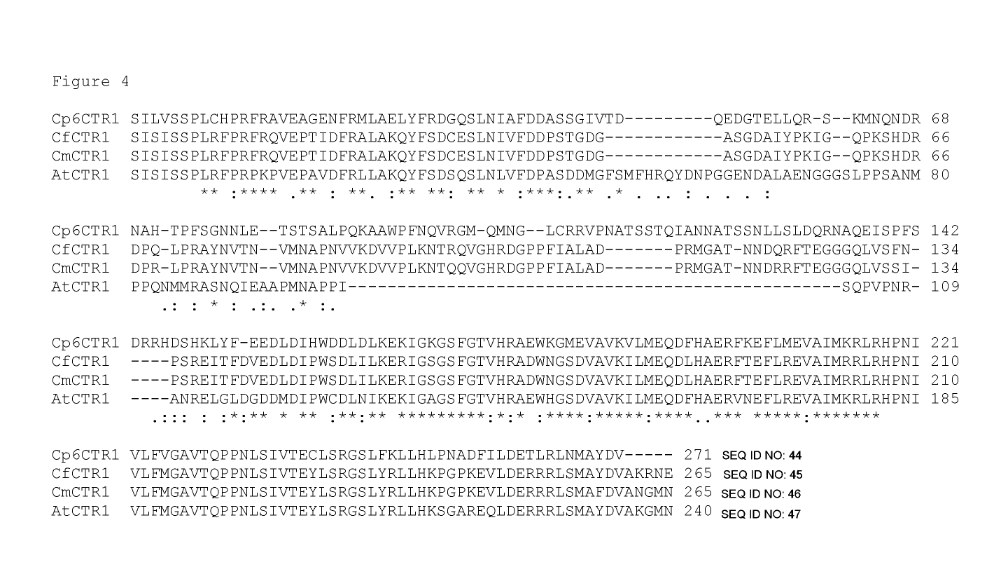

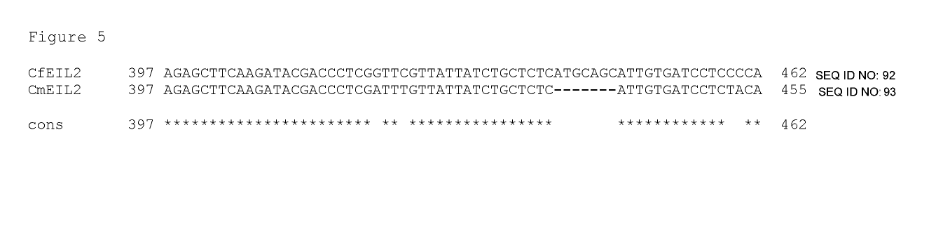

Degenerate primers compatible with CTR1 were used to identify partial sequences of CTR1 from Cp, Cf and Cm flower cDNA. During sequencing, small discrepancies were found which could not be explained as nucleotide errors but rather may have arisen from the presence of several very similar homologs of CTR1. The CTR1 transcripts cloned from Cp, Cf and Cm all appeared to represent a mix of closely related CTR1 homologs possibly arising from different genes. When translated to proteins, the sequence variation in the partial CTR1 accounted for 2-3 amino acid changes of a total of 265-271 amino acids (data not shown). Alignment of one representative from each putative Campanula CTR1 protein fragment with Arabidopsis CTR1 showed several conserved regions (FIG. 4).

The first attempts to identify EIL homologs from Campanula were done with degenerate primers using cDNA from flowers as template. Sequencing of the resulting DNA fragments revealed two EIL homologs EIL1 and EIL2 from CpB and Cf, whereas Cm seemed to contain EIL1 only. Surprisingly, sequencing of Cm gDNA using the same primer sets revealed a homolog partial EIL2 DNA sequence containing a 7 bp deletion when compared to the sequence of CfEIL2 (FIG. 5). New primers were designed subsequently to efficiently separate EIL1 and EIL2 during cloning reactions and expression analysis. This resulted in specific amplification of CpBEIL2 and CfEIL2, but no transcripts were found of CmEIL2 in flowers.

Expression Analysis of Putative ERS2, CTR1, EIL1 and EIL2 Homologues

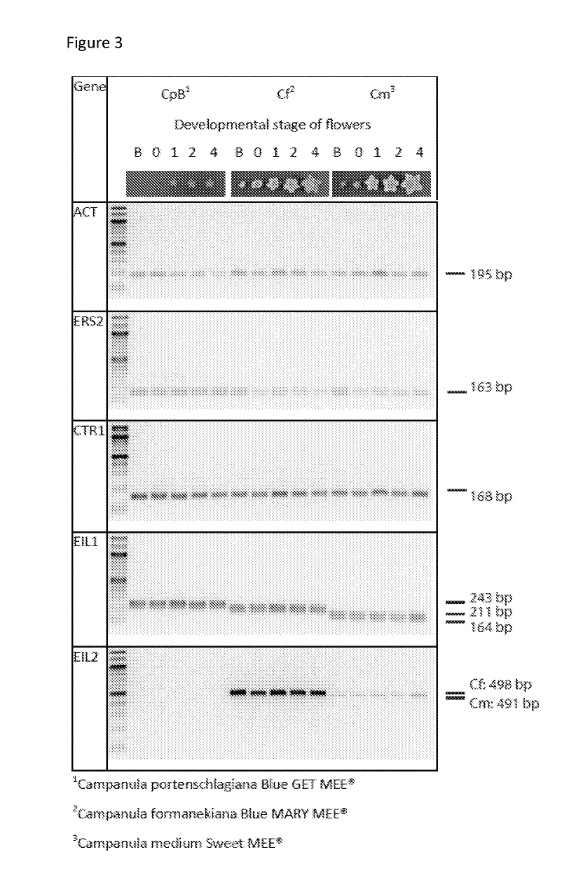

Expression levels of putative ERS2, CTR1, EIL1, and EIL2 were determined at five flower developmental stages (from bud to day 4) in CpB, Cf, and Cm (FIG. 3). ERS2, CTR1, and EIL1 were constitutively expressed in all developmental stages investigated in all cultivars (FIG. 3). In contrast, the expression of EIL2 was high in Cf whereas the transcript was absent in Cm (FIG. 3). An EIL2 homolog was not detected in CpB. Expression patterns of ERS, CTR1 and EIL1 in CpW and CpD were similar to those obtained in CpB (data not shown).

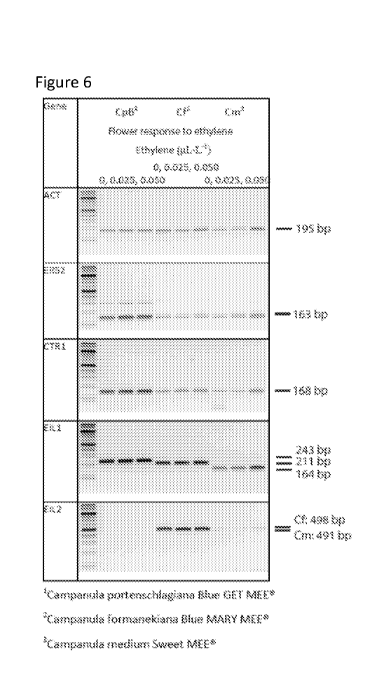

To determine whether ERS2, CTR1, EIL1, and EIL2 are regulated by ethylene, expression levels were characterized in young (1-day old) flowers exposed to 0.025 μL/L and 0.05 μL/L ethylene for 24 hours. No differences in expression levels in response to ethylene were observed for ERS2, CTR1, or EIL1 (FIG. 6) in any of the Campanula species examined. In Cf, a constitutive high expression of EIL2 was observed whereas no expression was detected in Cm in any of the ethylene treatments (FIG. 6). An EIL2 homolog was not detected in CpB.

The EIL2 Deletion is Specific for Campanula medium

Alignment of the partial putative EIL2 protein sequences from Campanula with EIL sequences from other plants showed the presence of three conserved EIL domains consisting of two basic amino acids domains (BD I and BD II) and the proline-rich region (PR). High homology was found within EIL from Campanula species except for CmEIL2. This putative protein sequence exhibited high homology to EILs in other plants, however the sequence following downstream of the frame shift caused by the 7 nucleotide deletion in the corresponding DNA was only observed in Cm. In support of a deletion in the CmEIL2 reading frame is the fact that the frame shift occurs in the conserved SALM motif present in most EILs and that omission of the deletion from the CmEIL2 reading frame results in a putative protein that perfectly align with other EIL2 proteins (FIG. 7). Phylogenetic analysis of partial EIL proteins from other plant species showed that EILs within Campanula species are more identical to each other than to orthologous found in other plants (FIG. 10). Comparison with EIL sequences from other plants supported this in general (FIG. 10). Different cultivars or lines of Campanula medium have also been tested and characterised according to the above described procedures. Some of these cultivars, Campanula medium_AP, Campanula medium_6, Campanula medium_80, and Campanula medium_123, comprised a polypeptide motif sequence of SASLI, and a downstream part according to the invention, whereas other species, Campanula medium_64, Campanula medium_11, Campanula medium_3, and Campanula medium_30, contained the polypeptide motif sequence SSLI, and a downstream part according to the invention, cf. FIGS. 8 and 9. These cultivars exhibited the same insensitivity towards exposure to ethylene as their Campanula medium.

### REFERENCES

Alberta Environment. (2003), Assessment Report on Ethylene for Developing Ambient Air Quality Objectives, http://environment.gov.ab.ca/info/library/6721.pdf.
Alonso, J. M., Hirayama, T., Roman, G., Nourizadeh, S. and Ecker, J. R. (1999), EIN2, a bifunctional transducer of ethylene and stress responses in Arabidopsis. Science, 284, 2148.
Alonso, J. M., Stepanova, A. N., Solano, R., Wisman, E., Ferrari, S., Ausubel, F. M. and Ecker, J. R. (2003) Five components of ethylene-response pathway identified in a screen for weak ethylene-insensitive mutants in Arabisopsis. Proc Natl Acad Sci USA, 100, 2992-2997.
Belhaj, K., Chaparro-Garcia, A., Kamoun, S. and Nekrasov V. (2013) Plant genome editing made easy: targeted mutagenesis in model and crop plants using the CRISPR/Cas system. Plant Methods, 9, 39.
Beyer, E. M. (1976) A Potent Inhibitor of Ethylene Action in Plants, Plant Physiol, 58, 268-271.
Binder, B. M., Walker, J. M., Gagne, J. M., Emborg, T. J., Hemmann, G., Bleecker, A. B. and Vierstrab, R. D. (2007) The Arabidopsis EIN3 Binding F-Box Proteins EBF1 and EBF2 Have Distinct but Overlapping Roles in Ethylene Signalling. Plant Cell 19, 509-523.
Bleecker, A. B., Estelle, M. A., Somerville, C. and Kende, H. (1988) Insensitivity to ethylene conferred by dominant mutation in Arabidopsis thaliana. Science, 241, 1-25.
Cameron, A. C. and Reid, M. S. (2001) 1-MCP blocks ethylene-induced petal abscission of Pelargonium peltatum but the effect is transient. Postharvest Biol Tec, 22, 169-177.
Chao, Q., Rothenberg, M., Solano, R., Roman, G., Terzaghi, W. and Ecker, J. R. (1997) Activation of the ethylene gas response pathway in Arabidopsis by nuclear protein ETHYLENE-INSENSITIVE3 and related proteins. Cell, 89, 1133-1144.
Chang, H., Jones, M. L., Banowets, G. M. and Clarke, D. G. (2003) Overproduction of cytokinins in petunia flowers transformed with PSAG12-IPT delays corolla senescence and decreases sensitivity to ethylene. Plant Physiol, 132: 539-544.
Chang, C., Kwok, S. F., Bleecker, A. B. and Meyerowitz, E. M. (1993) Arabidopsis ethylene-response gene ETR1: similarity of product to two-component regulators. Science, 262, 539-544.
Chen, S. Y., Tsai, H. C. Raghu, R. Do, Y. Y. and Huang, P. L. (2011) cDNA cloning and functional characterization of ETHYLENE INSENSITIVE 3 orthologues from Oncidium Gower Ramsey involved in flower cutting and pollinia cap dislodgement. Plant Physiol Bioch, 49, 1209-1219.
Ciardi, J., Barry, K., Shibuya, K. Nourizadeh, S., Ecker, J., Klee, H. and Clark, D. (2003) Increased flower longevity in petunia through manipulation of ethylene signaling genes. Proceedings of the NATO Advanced Research Workshop on Biology and Biotechnology of the Plant Hormone Ethylene III. (Vendrell, M., Klee, H., Pech, J. C. and Romojaro, F., eds) pp. 370-372. Amsterdam: IOS Press.
Clarke, K. L., Larsen, P. B., Wang, X., and Chang, C. (1998) Accociation of Arabidopsis CTR1 Raf-like kinase with the ETR1 and ERS ethylene receptors. Proc Natl Acad Sci USA, 95, 5401-5406.
Clevenger, D., Barrett, J., Klee, H., and Clark, D. (2004) Factors affecting seed production in transgenic ethylene-insensitive petunia. J Am Soc Hortic Sci, 129, 401-406.
Finlayson, S. A. and Reid, D. M. (1994) Influence of CO2 on ACC oxidase activity from roots of sunflower (Helianthus annuus) seedlings. Phytochemistry, 35, 847-851.
Flora-Dania marketing A/S, (2013), Top 10 over danske kulturer 2011, http://floradania.dk/dk/branchenyt/top-14/.
Flora Holland, (2011), Kengetallen 2011, (pdf retrieved online Feb. 27, 2013).
Gamble, R. L., Coonfield, M. L. and Schaller, G. E. (1998) Histidine kinase activity of the ETR1 ethylene receptor from Arabidopsis. Proc Natl Acad Sci USA, 95, 7825-7829.
Grossmann, K. and Hansen, H. (2001) Ethylene-triggered abscisic acid: A principle in plant growth regulation? Plant Physiol, 113, 9-14.
Gubrium, E., Clevanger, D., Clarke, D., Barrett, J. and Nell, T. (2000) Reproduction and horticultural performance of transgenic ethylene-insensitive Petunias. J Am Soc Hortic Sci, 125, 277-281
Guo, H and Ecker, J. R. (2003) Plants responses to ethylene gas are mediated by SCF (EBF1/EBF2)-dependent proteolysis of EIN3 transcription factor. Cell, 115, 667-677.
Heywood, V. H., Brummitt, R. K., Culham, A. and Seberg, O. (2007) Flowering plant families of the world, Royal Botanic Gardens: Kew.

Hu, H-L., Do, Y-Y. and Huang, P-L. (2012) Expression profiles of a MhCTR1 gene in relation to banana fruit ripening. Plant Physiol Bioch, 56, 47-55.
Hua, J., Sakai, H., Nourizadeh, S., Chen, Q. G., Bleecker, A. B., Ecker, J. R. and Meyerowitz, E. M. (1998) EIN4 and ERS2 are Members of the Putative Ethylene Receptor Gene Family in Arabidopsis. Plant Cell, 10, 1321-1332.
Hua, J., Chang, C., Sun, Q. and Meyerowitz, E. M. (1995) Ethylene insensitivity conferred by Arabidopsis ERS gene. Science, 269, 1712-1714.
Hua, J. and Meyerowitz, E. M. (1998) Ethylene responses are negatively regulated by a receptor gene family in Arabidopsis thaliana. Cell, 94, 261-71.
Huang, W-F., Huang, P-L. and Do, Y-Y. (2007) Ethylene receptor transcript accumulation patterns during flower senescence in Oncidium ‘Gower Ramsey’ as affected by exogenous ethylene and pollinia cap dislodgement. Postharvest Biol Tech, 44, 97-94.
Iordachescu, M. and Verlinden, S. (2005) Transcriptional Regulation of three EIN3-like genes of carnation (Dianthus caryophyllus L. cv. Improved White Sim) during flower development and upon wounding, pollination, and ethylene exposure. J Exp Bot, 2011-2018.
Kato, M., Shimizu, H., Onozaki, T., Tanikawa, N., Ikeda, H., Hisamatsu, T., Ichimura, K., (2002), Role of Ethylene in Senescence of Pollinated and Unpollinated Campanula medium Flowers. J Jpn Soc Hortic Sci, 71(3), 285-387.
Kieber, J. J., Rothenberg, M., Roman, G., Feld mann, K. A., and Ecker, J. R., (1993), CTR1, a negative regulator of ethylene response pathway in Arabidopsis, encodes a member of the raf family of protein kinases. Cell, 72, 427-441.
Larkin, M. A., Blackshields, G., Brown, N. P., Chenna, R., McGettigan, P. A., McWilliam, H. Valentin, F., Wallace, I. M., Wilm, A., Lopez, R., Thompson, J. D., Gibson, T. J. and Higgins, D. G. (2007) Clustal W and Clustal X version 2.0. Bioinformatics, 23(21), 2947-2948.
Lawton, K. A., Huang, B., Goldsbrough, P. B. and Woodson, W. R. (1989) Molecular cloning and characterization of senescence-related genes from carnation flower petals. Plant Physiol, 90, 690-696.
Leclercq, J., Adams-Phillips, L. C., Zegzouti, H., Jones, B., Latche′, A., Giovannoni, J. J., Pech, J-C. and Bouzayen, M. (2002) LeCTR1, a Tomato CTR1-Like Gene, DemonstratesEthylene Signaling Ability in Arabidopsis and Novel Expression Patterns in Tomato. Plant Physiol, 130, 1132-1142.
Lin, Z., Zhong, S. and Grierson, D. (2009) Recent advances in ethylene research, J Exp Bot, 60, 3311-3336.
Lusser, M., Parisi, C., Plan, D and Rodriguez-Cerezo, E. (2012) Deployment of new biotechnologies in plant breeding. Nat Biotechnol, 30, 231-239.
Laken, H., Clarke, J. L. and Müller, R. (2012) Review: Genetic engineering and sustainable production of ornamentals: current status and future directions. Plant Cell Rep, 31, 1141-1157.
Ma, N., Tan, H., Liu, X. H., Xue, J. Q., Li, Y. H. and Gao, J. P. (2006) Transcriptional regulation of ethylene receptor and CTR genes involved in ethylene-induced flower opening in cut rose (Rosa hybrida) cv. Samantha. J Exp Bot, 57, 2763-2773.
Mbeguie, D., Hubert, O., Fils-Lycaon, B., Chillet, M. and Baurens, F. C. (2008) EIN3-like gene expression during fruit ripening of Cavendish banana (Musa accuminata cv. Grande niane). Plant Physiol, 133, 435-488.
Moussatche, P. and Klee, H. J. (2004) Autophosphorylation activity of the Arabidopsis ethylene receptor multigene family. J Biol Chem, 279, 48734-48741.
Mudge, K. W. (1988) Effects of Ethylene on rooting. In: Adventitious root formation in cuttings, advances in plant science series, Vol 2, (Davis, T. D., Haissig, B. E. and Sankhla, N., eds) pp. 150-161. Portland: Dioscorides Press.
Müller, R. (2011) Physiology and genetics of plant quality improvement. Doctoral Dissertation, University of Copenhagen.
Müller, R., Andersen, A. S., and Serek, M. (1998) Differences in display life of miniature potted roses (Rosa hybrida L.), Sci Hortic-Amsterdam, 76, 59-71.
Muller R., Owen C. A., Xue, Z. T., Welander, M. and Stummann, B. (2003a) The transcription factor EIN3 is constitutively expressed in miniature roses with differences in postharvest life. J Hortic Sci Biotech, 78 (1):10-14.
Müller, R. Owen, C. A., Xu Z-T., Welander, M. and Stummann, B. M. (2003b) Genetic control of ethylene perception and signal transduction related to flower senescence. J Food Agric Environ, 1, 87-94.
Müller, R., Stumman, B. M. and Andersen, A. S. (2001a) Comparison of postharvest properties in closely related miniature rose cultivars (Rosa Hybrida L.). Sci Hortic-Amsterdam, 91, 325-337.
Müller, R. Stummann, B. M., Sisler, E. C and Serek, M. (2001b) Cultivar differences in resolution of ethylene production of miniature rose flowers (Rosa hybrida L.). Gartenbauwissenshaft.
Nell, T. A. (1992) Taking silver out of the longevity picture. Grower Talks, 6, 35-38.
Notredame, C., Higgins, D. G., and Heringa, J. (2000) T-Coffee: A novel method for multiple sequence alignments. J Mol Biol, 302, (205-217)
OECD SIDS, Ethylene, (http://www.inchem.org/documents/sids/sids/74851.pdf,).
Qiao, H., Shen, Z., Huang, S-S. C., Schmitz, R. J., Ulrich, M. A., Briggs, S. P. and Ecker, J. R. (2012) Processing and Subcellular Traficking of ER-Tethered EIN2 Control Response to Ethylene Gas. Science, 338, 390-393.
Rieu, I., Mariani, C. and Weterings, K. (2003) Expression analysis of five tobacco EIN3 family members in relation to tissue-specific ethylene responses. J Exp Bot, 54, 2239-2244.
Rodriguez, F. I., Esch, J. J., Hall, A. E., Binder, B. M., Schaller, G. E. and Bleecker, A. B. (1999) A copper cofactor for the ethylene receptor ETR1 from Arabidopsis. Science, 283, 996-998.
Roman, G., Lubarsky, B., Kieber, J. J., Rothenberg, M. and Ecker, J. R. (1995) Genetic-analysis of ethylene signal-transduction in Arabidopsis-thaliana —5 novel mutant loci integrated into stress-respons pathway. Genetics, 139, 1393.
Serek, M. and Reid, M. S. (1993) Anti-ethylene treatments for potted Christmas Cactus—Efficacy of inhibitors of ethylene action and biosynthesis. HortScience 28, 1180-1181.
Serek, M. and Reid, M. S. (2000) Ethylene and postharvest performance of potted kalanchoë. Postharvest Biol Tech, 18, 43-48.
Serek, M., Woltering, E. J., Sisler, E. C., Frello, S. and Sriskandarajah, S. (2006) Controlling ethylene responses in flowers at the receptor level. Biotechnol Adv, 24, 368-381.
Shaw, J., Chen, H., Tsai, M., Kuom, C. I. and Huangm, L. C. (2004) Extended flower longevity of Petunia hybrid plants transformed with boers, a mutated ERS gene of Brassica oleracea. Mol Breed, 9, 211-216.
Shibuya, K., Nagata, M., Tanikawa, N., Yoshioka, T., Hashiba, T. and Satoh, S. (2002) Comparison of mRNA levels of three ethylene receptors in senescing flowers of carnation (Dianthus caryphyllus L.). J Exp Bot, 53 (268), 399-406.
Shibuya, K., Berry, K. G., Ciardi, J. A., Loucas, H. M., Underwood, B. A., Noirizadeh, S., Ecker, J. R., Klee, H. J. and Clark, D. G. (2004) The central role of PhEIN2 in ethylene responses throughout plant development in petunia. Plant Physiol, 136, 2900-2912.
Shibuya, K., Clark, D. G. (2006) Ethylene: current status and future directions of using transgenic techniques to improve flower longevity of ornamental crops. J Crop Improv, 18, 391-412.
Sriskandarajah, S., Frello, S., Jørgensen, K. and Serek, M. (2004) Agrobacterium tumefaciens-mediated transformation of Campanula carpatica: factors affecting transformation and regeneration of transgenic shoots. Plant Cell Rep, 23, 59-63.
Sriskandarajah, S., Mibus, H. and Serek, M. (2007) Transgenic Campanula carpatica plants with reduced ethylene sensitivity, Plant Cell Rep, 26, 805-813
Sun, Y., Christensen, B., Liu, F, Wang, H. and Müller, R. (2009) Effects of ethylene and 1-MCP (1-methylcyclopropene) on bud and flower drop in mini Phalaenopsis cultivars. Plant Growth Regul, 59, 83-91.
Tieman, D. M., Ciardi, J. A., Taylor, M. G. and Klee, H. J. (2001) Members of the tomato LeEIL (EIN3-like) gene family are functionally redundant and regulate ethylene responses throughout plant development. Plant J, 26(1), 47-58.
Trivellini, A., Ferrante, A., Vernieri, P. and Serra, G. (2011) Effects of abscisic acid on ethylene biosynthesis and perception in Hibiscus rosa-sinensis L. flower development. J Exp Bot, 62 (15), 5437-5452.
Voet van Vormizeele, J. and Groth, G. (2008) Ethylene controls autophosphorylation of the histidine kinase domaine in ethylene receptor ETR1. Mol Plant, 1, 380-387.
Wang, Y., Zhang, C., Peiyi, J., Wang, X., Wang, W. and Dong, L. (2013) Isolation and expression analysis of three EIN3-like genes in tree peony (Paeonia suffruticosa). Plant Cell Tiss Org, 112, 181-190.
Wilkinson, J. Q., Lanahan, M. B., Clark, D. G., Bleecker, A. B., Chang, C., Meyerowitz, E. M. and klee, H. J. (1997) A dominant mutant receptor from Arabidopsis confers ethylene insensitivity in heterologous plants. Nat Biotechnol, 15, 444-447.
Woltering, E. J. (1987) Effects of ethylene on ornamental pot plants: a classification. Sci Hortic-Amsterdam, 31, 283-294.
Yokotani, N., Tamura, S., Nakano, R., Inaba, A. and Kubo, Y. (2003) Characterization of a novel tomato EIN3-like gene (LeEIL4). J Exp Bot, 54 (393), 2775-2776.
Zhou, Z., Dong, L., Jia, P. Y., Wang, W. R. and Wang, L. Y. (2010) Expression of ethylene receptor and transcription factor genes, and ethylene response during flower opening in tree peony (Paeonia suffruticosa). Plant Growth Regul, 62, 171-179.

1-13. (canceled)

14. A non-natural plant comprising a nucleic acid sequence selected from the group consisting of:
a nucleic acid sequence encoding an EIN/EIL polypeptide comprising an upstream part having at least 80% sequence identity with amino acids 1 to 143 of SEQ ID NO: 9, a polypeptide motif consisting of the amino acid sequence SxLy, with x being any natural amino acid and y being any natural amino acid, and a downstream part consisting of more than 15 amino acids and having below 50% identity to SEQ ID NO: 10;
a nucleic acid sequence encoding an EIN/EIL polypeptide having at least 70% identity to SEQ ID NO: 3, SEQ ID 11, SEQ ID NO: 12, SEQ ID 13, or SEQ ID NO: 14; and
a nucleic acid sequence having at least 85% identity to SEQ ID NO: 4, SEQ ID NO: 15, SEQ ID NO: 16, SEQ ID NO: 17, or SEQ ID NO: 18.

15. The non-natural plant according to claim 14, wherein x is A or S.

16. The non-natural plant according to claim 14, wherein y is selected from the group consisting of F, G, I and M.

17. The non-natural plant according to claim 14, which plant is selected from the group consisting of crops, vegetables, fruits and flowers.

18. The non-natural plant according to claim 14, which plant is selected from the group consisting of broccoli, tomato, petunia, carnations, orchids, Kalanchoë, Campanula and roses.

19. A method for preparing a plant cell having reduced sensitivity to ethylene, comprising:
identifying a EIL2 ortholog gene in a plant cell, which EIL2 ortholog gene encodes a protein having at least 70% sequence identity with SEQ ID NO: 5, 6, 7 or 8; and
disrupting or modifying the EIL2 ortholog gene in the plant cell to obtain a plant cell having a nucleic acid sequence selected from the group consisting of:
a nucleic acid sequence encoding an EIN/EIL polypeptide comprising an upstream part having at least 80% sequence identity with amino acids 1 to 143 of SEQ ID NO: 9, a polypeptide motif consisting of the amino acid sequence SxLy, with x being any natural amino acid and y being any natural amino acid, and a downstream part consisting of more than 15 amino acids and having below 50% identity to SEQ ID NO: 10;
a nucleic acid sequence encoding an EIN/EIL polypeptide having at least 70% identity to SEQ ID NO: 3, SEQ ID 11, SEQ ID NO: 12, SEQ ID 13, or SEQ ID NO: 14; and
a nucleic acid sequence having at least 85% identity to SEQ ID NO: 4, SEQ ID NO: 15, SEQ ID NO: 16, SEQ ID NO: 17, or SEQ ID NO: 18.

20. The method according to claim 19, wherein x is A or S.

21. The method according to claim 19, wherein y is selected from the group consisting of F, G, I and M.

22. The method according to claim 19, wherein the plant cell is obtained from the group of plants comprising crops, vegetables, fruits and flowers.

23. The method according to claim 19, wherein the plant cell is obtained from the group of plants comprising broccoli, tomato, petunia, carnations, orchids, Kalanchoe, Campanula and roses.

24. The method according to claim 19, wherein the plant cell is selected from the group consisting of protoplasts, gamete producing cells, and cells which regenerate into a whole plant.

25. A method for preparing a non-natural plant having reduced sensitivity to ethylene, comprising:
producing plants from plant cells according to claim 19; and
selecting plants that have a reduced sensitivity to ethylene.

26. A method of producing a transgenic plant having reduced sensitivity to ethylene, the method comprising the steps of:
providing a transformed plant cell by contacting a plant cell with a vector comprising a nucleic acid sequence selected from the list consisting of:
a nucleic acid sequence encoding an EIN/EIL polypeptide comprising an upstream part having at least 80% sequence identity with amino acids 1 to 143 of SEQ ID NO: 9, a polypeptide motif consisting of the amino acid sequence SxLy, with x being any natural amino acid and y being any natural amino acid, and a downstream part consisting of more than 15 amino acids and having below 50% identity to SEQ ID NO: 10;
a nucleic acid sequence encoding an EIN/EIL polypeptide having at least 70% identity to SEQ ID NO: 3, SEQ ID 11, SEQ ID NO: 12, SEQ ID 13, or SEQ ID NO: 14; and
a nucleic acid sequence having at least 85% identity to SEQ ID NO: 4, SEQ ID NO: 15, SEQ ID NO: 16, SEQ ID NO: 17, or SEQ ID NO: 18,
which nucleic acid sequence is operably associated with a promoter, and producing a plant from the transformed plant cell.

27. The method of producing a transgenic plant according to claim 26, wherein the vector further comprises one or more selectable marker genes.

28. The method according to claim 26, wherein x is A or S.

29. The method according to claim 26, wherein y is selected from the list consisting of F, G, I and M.

30. A product, or a processed product thereof, derived from the plant according to claim 14.

31. The product according to claim 30, which product is selected from the group consisting of flowers, seeds, leaves, branches, roots, and fruits.

32. The non-natural plant according to claim 17, wherein the fruits and flowers comprise climacteric fruits and climacteric flowers.

33. The method according to claim 22, wherein the fruits and flowers comprise climacteric fruits and climacteric flowers.


SEQUENCE LISTING (93 sequences)

SEQ ID NO: 1 - protein, 196 aa, Campanula formanekiana
  VARIANT (0)...(0): EIL1
ELERRMWRDRMLLRRLKEQSKGKEGVDSAKQRQSQEQARRKKMSRAQDGILKYMLKMMEVCKAQGFVYGIIPENGKPVSGASDNLRAWWKEKVRFDRNGPAAIAKYQADNSVPGKSEDCCTMGPTPHTLQELQDTTLGSLLSALMQHCDPPQRRFPLEKGVAPPWWPKGDEEWWAQLGLPKDQGPPPYKKPHDLKK

SEQ ID NO: 2 - protein, 200 aa, Campanula formanekiana
  VARIANT (0)...(0): EIL2
ELERRMWRDKLRLKRFKESNKGTKESCVDASKQRQSQEQARRKKMSRAQDGILKYMLKMMEVCKAQGFVYGIIPEKGKPVGGASDNLREWWKDKVKFDRNGPAAITKFQVDNSISGTDEGSNSIGPTPHTLQELQDTTLGSLLSALMQHCDPPQRRFPLEKGVPPPWWPIGTEEWWPQLGLQKDQGAGPPPYKKPHDLKK

SEQ ID NO: 3 - protein, 197 aa, Campanula medium
  VARIANT (0)...(0): EIL2
ELERRMWRDKLRLKRLKESNKGTKESCVDASKQRQSQEQARRKKMSRAQDGILKYMLKMMEVCKAQGFVYGIIPEKGKPVGGASDNLREWWKDKVKFDRNGPAAITKFQVDNSIPGTDEGSNSIGPTPHTLQELQDTTLDLLLSALIVILYRGVSLSRKVFRPRGGPRGRRNGGLNWVCRKNKGRAHRLTRSLMIRR

SEQ ID NO: 4 - DNA, 596 bp, Campanula medium
  misc_feature (0)...(0): EIL2 gDNA
agagctcgagaggaggatgtggagggacaaattgcgtctaaaaaggctcaaagaatcgaacaaggggacgaaggaatcgtgtgttgatgcatccaaacaacgccaatcccaggagcaagcaaggaggaagaaaatgtctagagctcaagatgggattttgaaatatatgttaaaaatgatggaagtttgtaaagctcaaggttttgtttacgggatcattcccgagaaagggaagcctgtgggcggggcgtccgacaaccttcgagagtggtggaaggataaagttaagtttgacaggaatggaccggctgccattacgaaattccaagtggataattctattccggggacggatgaggggagtaattcgattggccctaccccacatactctccaagagcttcaagatacgaccctcgatttgttattatctgctctcattgtgatcctctacagaggcgtttccctctcgagaaaggtgttccgcccccgtggtggcccacgaggacggaggaatggtggcctcaattgggtttgcagaaagaacaaggggcgggcccaccgccttacaagaagcctcatgatctgaagaaggc

SEQ ID NO: 5 - protein, 196 aa, Campanula medium
  VARIANT (0)...(0): EIL1
ELERRMWRDRMLLRRLKEQNKGKEGVDSAKQRQSQEQARRKKMSRAQDGILKYMLKMMEVCKAQGFVYGIIPENGKPVSGASDNLRAWWKEKVRFDRNGPAAIAKYQLDNSVPGKSEDCCTMGPTPHTLQELQDTTLGSLLSALMQHCDPPQRRFPLEKGVAPPWWPKGDEEWWAQLGLPKDQGPPPYKKPHDLKK

SEQ ID NO: 6 - protein, 197 aa, Campanula portenschlagiana
  VARIANT (0)...(0): EIL1
ELERRMWRDRMLLRRLKEQNKGKEGVVDNAKQRQSQEQARRKKMSRAQDGILKYMLKMMEVCKAQGFVYGIIPENGKPVSGASDNLRAWWKEKVRFDRNGPAAIAKYQADHSAPGKSEDCCTMASTPHTLQELQDTTLGSLLSALMQHCDPPQRRFPLEKGVAPPWWPNGDEEWWAQLGLPKDQGPPPYKKPHDLKK

SEQ ID NO: 7 - protein, 196 aa, Campanula portenschlagiana
  VARIANT (0)...(0): EIL2
ELERRMWRDRMLLRRLKEQNKGKEGVDNAKQRQSQEQARRKKMSRAQDGILKYMLKMMEVCKAQGFVYGIIPEKGKPASGASDNLRAWWKERVRFDRNGPAAITKYQADHSVPGESEDCCMVASTPHTLQELQDTTLGSLLSALMQHCDPPQRRFPLEKGVAPPWWPNGDEEWWPQLGLPKDQGPPPYKKPHDLKK

SEQ ID NO: 8 - protein, 628 aa, Arabidopsis Thaliana
MMFNEMGMCGNMDFFSSGSLGEVDFCPVPQAEPDSIVEDDYTDDEIDVDELERRMWRDKMRLKRLKEQDKGKEGVDAAKQRQSQEQARRKKMSRAQDGILKYMLKMMEVCKAQGFVYGIIPENGKPVTGASDNLREWWKDKVRFDRNGPAAITKYQAENNIPGIHEGNNPIGPTPHTLQELQDTTLGSLLSALMQHCDPPQRRFPLEKGVPPPWWPNGKEDWWPQLGLPKDQGPAPYKKPHDLKKAWKVGVLTAVIKHMFPDIAKIRKLVRQSKCLQDKMTAKESATWLAIINQEESLARELYPESCPPLSLSGGSCSLLMNDCSQYDVEGFEKESHYEVEELKPEKVMNSSNFGMVAKMHDFPVKEEVPAGNSEFMRKRKPNRDLNTIMDRTVFTCENLGCAHSEISRGFLDRNSRDNHQLACPHRDSRLPYGAAPSRFHVNEVKPVVGFPQPRPVNSVAQPIDLTGIVPEDGQKMISELMSMYDRNVQSNQTSMVMENQSVSLLQPTVHNHQEHLQFPGNMVEGSFFEDLNIPNRANNNNSSNNQTFFQGNNNNNNVFKFDTADHNNFEAAHNNNNNSSGNRFQLVFDSTPFDMASFDYRDDMSMPGVVGTMDGMQQKQQDVSIWF

SEQ ID NO: 9 - protein, 147 aa, Campanula medium
  VARIANT (0)...(0): EIL2 - upstream part including SALI
ELERRMWRDKLRLKRLKESNKGTKESCVDASKQRQSQEQARRKKMSRAQDGILKYMLKMMEVCKAQGFVYGIIPEKGKPVGGASDNLREWWKDKVKFDRNGPAAITKFQVDNSIPGTDEGSNSIGPTPHTLQELQDTTLDLLLSALI

SEQ ID NO: 10 - protein, 51 aa, Arabidopsis Thaliana
  VARIANT (0)...(0): AtEil1 - downstream part
QHCDPPQRRFPLEKGVSPPWWPNGNEEWWPQLGLPNEQGPPPYKKPHDLKK

SEQ ID NO: 11 - protein, 196 aa, Campanula medium
  VARIANT (0)...(0): Cultivar 64
LELERRMWTDKFHLKRLKESNKGTKESCVDASKQRQSQEQARRKKMSRAQDGILKYMLKMMEVCKAQGFVYGIIPEKGNPVGGASDNLREWWKDKVKFDRNGPAAITKFQVDNSIPGTDEGSNSIGPTPHTLQELQDTTLGSLLSSLIVILPRGVSLWRKVFRPRGCPRGRRNGGLNWVCRKNKGRAHRLTRSLMI

SEQ ID NO: 12 - protein, 159 aa, Campanula medium
  VARIANT (0)...(0): Cultivar 11
SCVDASKQRQSQEQARRKKMSRAQDGILKYMLKMMEVCKAQGFVYGIIPEKGNPVGGASDNLREWWKDKVKFDRNGPAAITKFQVDNSIPGTDEGSNSIGPTPHTLQELQDTTLGSLLSSLIVILPRGVSLWRKVFRPRGCPRGRRNGGLNWVCRRIKG

SEQ ID NO: 13 - protein, 163 aa, Campanula medium
  VARIANT (0)...(0): Cultivar 3
GTKESCVDASKQRQSQEQARRKKMSRAQDGILKYMLKMMEVCKAQGFVYGIIPEKGNPVGGASDNLREWWKDKVKFDRNGPAAITKFQVDNSIPGTDEGSNSIGPTPHTLQELQDTTLGSLLSSLIVILPRGVSLWRKVFRPRGCPRGRRNGGLNWVCRRIKG

SEQ ID NO: 14 - protein, 163 aa, Campanula medium; identical to SEQ ID NO: 13
  VARIANT (0)...(0): Cultivar 30

SEQ ID NO: 15 - DNA, 588 bp, Campanula medium
ttagagctagagaggaggatgtggacggacaaatttcatctaaaaaggctcaaagaatcgaacaaggggacgaaggaatcatgtgttgatgcatccaaacaacgccaatcccaggagcaagcaaggaggaagaaaatgtctagagctcaagatgggattttgaaatatatgttgaaaatgatggaagtttgtaaagctcaaggttttgtttacgggatcattcccgagaaagggaatcctgtgggcggggcgtccgacaaccttcgagagtggtggaaggataaagttaagtttgacaggaatggaccggctgccattacgaaattccaagtggataattctattccggggacggatgaggggagtaattcgattggccctaccccacatactctccaagagcttcaagatacgaccctcggttcgttattatcttctctcattgtgatcctccccagaggcgtttccctctggcgaaaggtgttccgcccccgtggttgcccacggggacggaggaatggtggcctcaattgggtttgcagaaagaacaaggggcgggcccaccgccttacaagaagcctcatgatc

SEQ ID NO: 16 - DNA, 479 bp, Campanula medium
tcatgtgttgatgcatccaaacaacgccaatcccaggagcaagcaaggaggaagaaaatgtctagagctcaagatgggattttgaaatatatgttgaaaatgatggaagtttgtaaagctcaaggttttgtttacgggatcattcccgagaaagggaatcctgtgggcggggcgtccgacaaccttcgagagtggtggaaggataaagttaagtttgacaggaatggaccggctgccattacgaaattccaagtggataattctattccggggacggatgaggggagtaattcgattggccctaccccacatactctccaagagcttcaagatacgaccctcggttcgttattatcttctctcattgtgatcctccccagaggcgtttccctctggcgaaaggtgttccgcccccgtggttgcccacggggacggaggaatggtggcctcaattgggtttgcagaaggatcaaggggcg

SEQ ID NO: 17 - DNA, 492 bp, Campanula medium
ggggacgaaggaatcatgtgttgatgcatccaaacaacgccaatcccaggagcaagcaaggaggaagaaaatgtctagagctcaagatgggattttgaaatatatgttgaaaatgatggaagtttgtaaagctcaaggttttgtttacgggatcattcccgagaaagggaatcctgtgggcggggcgtccgacaaccttcgagagtggtggaaggataaagttaagtttgacaggaatggaccggctgccattacgaaattccaagtggataattctattccggggacggatgaggggagtaattcgattggccctaccccacatactctccaagagcttcaagatacgaccctcggttcgttattatcttctctcattgtgatcctccccagaggcgtttccctctggcgaaaggtgttccgcccccgtggttgcccacggggacggaggaatggtggcctcaattgggtttgcagaaggatcaaggggcg

SEQ ID NO: 18 - DNA, 492 bp, Campanula medium; identical to SEQ ID NO: 17

SEQ ID NO: 19 - protein, 147 aa, Arabidopsis thaliana
  VARIANT (0)...(0): ATEil1, upstream part, including SALM motif
ELEKRMWRDKMRLKRLKEQQSKCKEGVDGSKQRQSQEQARRKKMSRAQDGILKYMLKMMEVCKAQGFVYGIIPEKGKPVTGASDNLREWWKDKVRFDRNGPAAIAKYQSENNISGGSNDCNSLVGPTPHTLQELQDTTLGSLLSALM

SEQ ID NO: 20 - DNA, 20 bp, Artificial Sequence
  ERS2 forward primer
tgtggaagttgttgctgacc

SEQ ID NO: 21 - DNA, 22 bp, Artificial Sequence
  ERS2 reverse primer
agctaggaaatcattacgagca

SEQ ID NO: 22 - DNA, 20 bp, Artificial Sequence
  CTR1 forward primer
cgaattgccaarggmtgtaa

SEQ ID NO: 23 - DNA, 21 bp, Artificial Sequence
  CTR1 reverse primer
attcatyccgtttgccacatc

SEQ ID NO: 24 - DNA, 21 bp, Artificial Sequence
  EIL1 forward primer
wgagctmgagaggaggatgtg

SEQ ID NO: 25 - DNA, 21 bp, Artificial Sequence
  EIL1 reverse primer
gccttcttcagatcatgaggc

SEQ ID NO: 26 - DNA, 22 bp, Artificial Sequence
  EIL2 forward primer
ggggacgaaggaatcatgtgtt

SEQ ID NO: 27 - DNA, 22 bp, Artificial Sequence
  EIL2 reverse primer
cgccccttgatccttctgcaaa

SEQ ID NO: 28 - DNA, 20 bp, Artificial Sequence
  ACTIN forward primer
gcaggacgtgatctgactga

SEQ ID NO: 29 - DNA, 21 bp, Artificial Sequence
  CTR1 forward primer
gttcgatttgggattgacagg

SEQ ID NO: 30 - DNA, 24 bp, Artificial Sequence
  CTR1 reverse primer
ccaatgacctgaaatcaatggttg

SEQ ID NO: 31 - DNA, 20 bp, Artificial Sequence
  CpBEIL1 forward primer
gcgaggactgctgtacgatg

SEQ ID NO: 32 - DNA, 20 bp, Artificial Sequence
  CpBEIL2 forward primer
gtgaggactgctgtatggtt

SEQ ID NO: 33 - DNA, 19 bp, Artificial Sequence
  CfEIL1 forward primer
acctcacacattgcaggaa

SEQ ID NO: 34 - DNA, 17 bp, Artificial Sequence
  CfEIL2 forward primer
cagaggcgtttccctct

SEQ ID NO: 35 - DNA, 21 bp, Artificial Sequence
  CmEIL1 forward primer
cagctttgatgcagcattgtg

SEQ ID NO: 36 - DNA, 22 bp, Artificial Sequence
  CmEIL2 forward primer
ctgctctcattgtgatcctcta

SEQ ID NO: 37 - DNA, 22 bp, Artificial Sequence
  ACTIN reverse primer
gggaacatrgttgawccaccac

SEQ ID NO: 38 - protein, 54 aa, Artificial Sequence
  CfERS2
CGVVADQVAVALSHAAILEESMRARDQLMEQNIALDLARQEAEMAVRARNDFLA

SEQ ID NO: 39 - protein, 54 aa, Artificial Sequence
  CmERS2
VEVVADQVAVALSHAAILEESMRARDQLMEQNIALDLARQEAEMAVRARNDFLA

SEQ ID NO: 40 - protein, 53 aa, Artificial Sequence
  Cp6ERS2s
GVVADQVAVALSHAAILEESMRARDQLMEQNIALDLARQEAEMAICARNDFLA

SEQ ID NO: 41 - protein, 54 aa, Artificial Sequence
  Cp6ERSla
CGVVADQVAVALSHAAILEESMRARDQLMEQDIALDLARQEAELAIRARNDFLA

SEQ ID NO: 42 - protein, 54 aa, Artificial Sequence
  Cp6ERSlb
CGVVADQVAVALSHATILEESMRARDQLMEQNIALDLARQEAELAIRARNDFLA

SEQ ID NO: 43 - protein, 54 aa, Artificial Sequence
  AtERS2
VKVVADQVAVAISHAVILEESQLMREKLAEQNRALQVARENALRANQAKAAFEQ

SEQ ID NO: 44 - protein, 271 aa, Artificial Sequence
  Cp6CTR1
SILVSSPLCHPRFRAVEAGENFRMLAELYFRDGQSLNIAFDDASSGIVTDQEDGTELLQRSKMNQNDRNAHTPFSGNNLETSTSALPQKAAWPFNQVRGMQMNGLCRRVPNATSSTQIANNATSSNLLSLDQRNAQEISPFSDRRHDSHKLYFEEDLDIHWDDLDLKEKIGKGSFGTVHRAEWKGMEVAVKVLMEQDFHAERFKEFLMEVAIMKRLRHPNIVLFVGAVTQPPNLSIVTECLSRGSLFKLLHLPNADFILDETLRLNMAYDV

SEQ ID NO: 45 - protein, 265 aa, Artificial Sequence
  CfCTR1
SISISSPLRFPRFRQVEPTIDFRALAKQYFSDCESLNIVFDDPSTGDGASGDAIYPKIGQPKSHDRDPQLPRAYNVTNVMNAPNVVKDVVPLKNTRQVGHRDGPPFIALADPRMGATNNDQRFTEGGGQLVSFNPSREITFDVEDLDIPWSDLILKERIGSGSFGTVHRADWNGSDVAVKILMEQDLHAERFTEFLREVAIMRRLRHPNIVLFMGAVTQPPNLSIVTEYLSRGSLYRLLHKPGPKEVLDERRRLSMAYDVAKRNE

SEQ ID NO: 46 - protein, 265 aa, Artificial Sequence
  CmCTR1
SISISSPLRFPRFRQVEPTIDFRALAKQYFSDCESLNIVFDDPSTGDGASGDAIYPKIGQPKSHDRDPRLPRAYNVTNVMNAPNVVKDVVPLKNTQQVGHRDGPPFIALADPRMGATNNDRRFTEGGGQLVSSIPSREITFDVEDLDIPWSDLILKERIGSGSFGTVHRADWNGSDVAVKILMEQDLHAERFTEFLREVAIMRRLRHPNIVLFMGAVTQPPNLSIVTEYLSRGSLYRLLHKPGPKEVLDERRRLSMAFDVANGMN

SEQ ID NO: 47 - protein, 240 aa, Artificial Sequence
  AtCTR1
SISISSPLRFPRPKPVEPAVDFRLLAKQYFSDSQSLNLVFDPASDDMGFSMFHRQYDNPGGENDALAENGGGSLPPSANMPPQNMMRASNQIEAAPMNAPPISQPVPNRANRELGLDGDDMDIPWCDLNIKEKIGAGSFGTVHRAEWHGSDVAVKILMEQDFHAERVNEFLREVAIMKRLRHPNIVLFMGAVTQPPNLSIVTEYLSRGSLYRLLHKSGAREQLDERRRLSMAYDVAKGMN

SEQ ID NO: 48 - protein, 108 aa, Artificial Sequence
  CfEIL1
ELERRMWRDRMLLRRLKEQSKGKEGVDSAKQRQSQEQARRKKMSRAQDGILKYMLKYMLKMMEVCKAQGFVYGIIPENGKPVSGASDNLRAWWKEKVRFDRNGPAAIA

SEQ ID NO: 49 - protein, 108 aa, Artificial Sequence
  CmEIL1
ELERRMWRDRMLLRRLKEQNKGKEGVDSAKQRQSQEQARRKKMSRAQDGILKYMLKYMLKMMEVCKAQGFVYGIIPENGKPVSGASDNLRAWWKEKVRFDRNGPAAIA

SEQ ID NO: 50 - protein, 154 aa, Artificial Sequence
  CpBEIL1
ELERRMWRDRMLLRRLKEQNKGKEGVVDNAKQRQSQEQARRKKMSRAQDGILKYMLKYMLKMMEVCKAQGFVYGIIPENGKPVSGASDNLRAWWKEKVRFDRNGPAAIAKYQADHSAPGKSEDCCTMASTPHTLQELQDTTLGSLLSALMQHCD

SEQ ID NO: 51 - protein, 153 aa, Artificial Sequence
  CpBEIL2
ELERRMWRDRMLLRRLKEQNKGKEGVDNAKQRQSQEQARRKKMSRAQDGILKYMLKYMLKMMEVCKAQGFVYGIIPEKGKPASGASDNLRAWWKERVRFDRNGPAAITKYQADHSVPGESEDCCMVASTPHTLQELQDTTLGSLLSALMQHCD

SEQ ID NO: 52 - protein, 153 aa, Artificial Sequence
  MdEIL1
ELEKRMWRDRMLLKRLKEQTKGKEGVDNARQRQSQEQARRKKMSRAQDGILKYMLKYMLKMMEVCKAQGFVYGIIPEKGKPVSGASDNLRAWWKEKVRFDRNGPAAISKYQADHSIPGKNEDFSAVASTPHTLQELQDTTLGSLLSALMQHCN

SEQ ID NO: 53 - protein, 153 aa, Artificial Sequence
  MdEIL3
ELEKRMWRDRMLLKRLKEQTKGKEGVDNARQRQSQEQARRKKMSRAQDGILKYMLKYMLKMMEVCKAQGFVYGIIPEKGKPVSGASDNLRAWWKEKVRFDRNGPAAISKYQADHSIPGKNEHFSAVASTPHTLQELQDTTLGSLLSALMQHCN

SEQ ID NO: 54 - protein, 153 aa, Artificial Sequence
  MdEIL2
ELERRMWRDRMLLKRLREQTKGKERVDNARQRQSQEQARRKKMSRAQDGILKYMLKYMLKMMEVCKAQGFVYGIIPEKGKPVSGASDNLRAWWKEKVRFDRNGPAAISKYQADHSIPGKNEDCSAVVSTPHTLQELQDTTLGSLLSALMQHCD

SEQ ID NO: 55 - protein, 155 aa, Artificial Sequence
  PsEIL3
QLERRMWRDRVLLKRLKEQNVKGAKEGVDIVKQRQSQEQARRKKMSRAQDGILKYMLKYMLKMMEVCKAQGFVYGIIPEKGKPVSGASDNLRPWWKEKVRFDRNGPAAIAKYQADHSISGMNEDCSTLACTPHTLQELQDTTLGSLLSALMQHCD

SEQ ID NO: 56 - protein, 155 aa, Artificial Sequence
  PiEIN3
QLERRMWRDRVLLKRLKEQNVKGAKEGVDIVKQRQSQEQARRKKMSRAQDGILKYMLKYMLKMMEVCKAQGFVYGIIPEKGKPVSGASDNLRPWWKEKVRFDRNGPAAIAKYQADHSIPGMNEDCSTLACTPHTLQELQDTTLGSLLSALMQHCD

SEQ ID NO: 57 - protein, 155 aa, Artificial Sequence
  DcEIL1
ELERRMWRDKMLLKRLKEQNKDRCREGIDNVKKRQSQEQARRKKMSRAQDGILKYMLKYMLKMMEVCKAQGFVYGIIPEKGKPVSGASDNLRAWWKDKVRFDRNGPAAIAKYQADHLVQGMDEDCTAMGSTPHTLQELQDTTLGSLLSALMQHCD

SEQ ID NO: 58 - protein, 155 aa, Artificial Sequence
  DcEIL2
ELERRMWRDKMLLKRLKEQNKDRCREWIDNVKKRQSQEQARRKKMSRAQDGILKYMLKYMLKMMEVCKAQGFVYGIIPEKGKPVSGASDNLRAWWKDKVRFDRNGPAAIAKYQADHLVQGMDEDCTAMGSTPHTLQEFQDTTLGSLLSALMQHCD

SEQ ID NO: 59 - protein, 155 aa, Artificial Sequence
  DcEIL4
ELEQRMWRDKMLLRRLKEQTKDKCATEVDCGKKHQSQEQARRKKMSRAQDGILKYMLKYMLKMMEVCNAQGFVYGIIPEKGKPVSGASDNLRAWWKEKVRFDRNGPAAIAKYQADHSVPGMDEDCSATGSTPHTLHELQDTTLGSLLSALMQHCD

SEQ ID NO: 60 - protein, 155 aa, Artificial Sequence
  CfEIL2
ELERRMWRDKLRLKRFKESNKGTKESCVDASKQRQSQEQARRKKMSRAQDGILKYMLKYMLKMMEVCKAQGFVYGIIPEKGKPVGGASDNLREWWKDKVKFDRNGPAAITKFQVDNSISGTDEGSNSIGPTPHTLQELQDTTLGSLLSALMQHCD

SEQ ID NO: 61 - protein, 155 aa, Artificial Sequence
  CmEIL2
ELERRMWRDKLRLKRLKESNKGTKESCVDASKQRQSQEQARRKKMSRAQDGILKYMLKYMLKMMEVCKAQGFVYGIIPEKGKPVGGASDNLREWWKDKVKFDRNGPAAITKFQVDNSIPGTDEGSNSIGPTPHTLQELQDTTLDLLLSALIVILY

SEQ ID NO: 62 - protein, 153 aa, Artificial Sequence
  PsEIL2
ELERRMWRDKMRLKRLKEQGKSKEGVDSTKQRQSQEQARRKKMSRAQDGILKYMLKYMLKMMEVCKAQGFVYGIIPEKGKPVGGASDNLREWWKDKVKFDRNGPAAIAKYQADNSIPGNNEGCNMAGPTSHTLQELQDTTLGSLLSALMQHCE

SEQ ID NO: 63 - protein, 153 aa, Artificial Sequence
  AtEIN3.3
ELERRMWRDKMRLKRLKEQDKGKEGVDAAKQRQSQEQARRKKMSRAQDGILKYMLKYMLKMMEVCKAQGFVYGIIPENGKPVTGASDNLREWWKDKVRFDRNGPAAITKYQAENNIPGIHEGNNPIGPTPHTLQELQDTTLGSLLSALMQHCD

SEQ ID NO: 64 - protein, 153 aa, Artificial Sequence
  VrEIL1
ELEKRMWRDKVRHKRLKEQQKAKEGTDAVKQRQSQEQARRKKMSRAQDGILKYMLKYMLKMMEVCKAQGFVYGIIPEKGKPVTGASDNLREWWKDKVRFDRNGPAAIAKYQADNAIPGKNDGCNSIGPTPHTLQELQDTTLGSLLSALMQHCD

SEQ ID NO: 65 - protein, 153 aa, Artificial Sequence
  VrEIL2
ELEHRMWKDKMLLKRLKEQSKSKEGIDAVKQRQSQEQARRKKMSRAQDGILKYMLKYMLKMMEVCKAQGFVYGIIPEKGKPVTGASDNLREWWKDKVRFDRNGPAAITKYQADNAIPGRNDGCNSIGPTPHTLQELQDTTLGSLLSALMQHCD

SEQ ID NO: 66 - protein, 155 aa, Artificial Sequence
  AtEIL1
ELEKRMWRDKMRLKRLKEQQSKCKEGVDGSKQRQSQEQARRKKMSRAQDGILKYMLKYMLKMMEVCKAQGFVYGIIPEKGKPVTGASDNLREWWKDKVRFDRNGPAAIAKYQSENNISGGSNDCNSLVGPTPHTLQELQDTTLGSLLSALMQHCD

SEQ ID NO: 67 - protein, 153 aa, Artificial Sequence
  DcEIL3
ELERRMWRDKMRLKRLKDQSKVKEGVDVAKQRQSQEQARRKKMSRAQDGILKYMLKYMLKMMEVCKAQGFVYGIIPEKGKPVTGASDNLREWWKEKVRFDRNGPAAIAKYEADNLIFMNSNAGDSIRSSAHALQELQDTTLGSLLSALMQHCD

SEQ ID NO: 68 - protein, 155 aa, Artificial Sequence
  PeEIN3
ELERRIWRYRLLVKQRKEQQNTKNRDFSDSVKHRQSQEQARRKKMSRAQDGILKYMLKYMLKMMEVCKAQGFVYGIIPEKGKPVSGASDNLRGWWKEKVRFDRNGPAAIAKYQVDNGIQSPNSELNSRFISPHSLQELQDTTLGSLLSALMQHCD

SEQ ID NO: 69 - protein, 155 aa, Artificial Sequence
  OhEIL2
ELERRMWRDRLRHKRLKEKQNAKNRDHGDSLEHRQSQEQARRKKMSRAQDGILKYMLKYMLKMMEVCKAQGFVYGIIPEKGKPVGGASDNLRAWWKEKVRFDRNGPAAIAKYQTDNGIQSTNNELNSRLVSPHSLQELQDTTLGSLLSALMQHCD

SEQ ID NO: 70 - protein, 155 aa, Artificial Sequence
  OhEIL1
ELERRMWRDRLRLKRLKEQQQNKNKELGDSTKQRQSQEQARRKKMSRAQDGILKYMLKYMLKMMEVCKAQGFVYGIIPEKGKPVSGASDNLRGWWKEKVRFDRNGPAAIAKYQADNGIRSLNNEQNSKPVSPYSLQELQDTTLGSLLSALMQHCD

SEQ ID NO: 71 - protein, 163 aa, Artificial Sequence
  ZmEIL1
ELERRMWRDRMKLKRLRELQQSRGKDPMASGGGLADGSSKPRQSQEQARRKKMSRAQDGILKYMLKYMLKMMEVCRAQGFVYGIIPEKGKPVSGASDNLRAWWKEKVRFDRNGPAAIAKYQADNAVPGAENELASGAASPHSLQELQDTTLGSLLSALMQHCD

SEQ ID NO: 72 - protein, 107 aa, Artificial Sequence
  MaEIL1p
QDGILKYMLKYMLKMMEVCNAQGFVYGIIPEKGKPVSGASDNLRGWWKEKVRFDRNGPAAIAKYQADNAVPGSGSEINSGTASPHSLQELQDTTLGSLLSALMQHCD

SEQ ID NO: 73 - protein, 107 aa, Artificial Sequence
  MaEIL3p
QDGILKYMLKYMLKMMEVCKAQGFVYGIIPEKGKPVSGASDNLRGWWKEKVRFDRNGPAAIAKYQADNTIPGSNNEVVSSTVSPHSLQELQDTTLGSLLSALMQHCD

SEQ ID NO: 74 - protein, 155 aa, Artificial Sequence
  MaEIL4
ELERRMWRDRMRLKRLREQQQNKNKEQGDTAKQHQSQEQARRKKMSRAQDGILKYMLKYMLKMMEVCQAQGFVYGIIPEKGKPVSGASDNLRGWWKEKVRFDRNGPAAIAKYQADNAIPGSSSEAIPGTVSPHSLQELQDTTLGSLLSALMQHCD

SEQ ID NO: 75 - protein, 157 aa, Artificial Sequence
  AtEIL2
ELEKKIWRDKQRLKRLKEMAKNGLGTRLLLKQQHDDFPEHSSKRTMYKAQDGILKYMSKYMSKTMERYKAQGFVYGIVLENGKTVAGSSDNLREWWKDKVRFDRNGPAAIIKHQRDINLSDGSDSGSEVGDSTAQKLLELQDTTLGALLSALFPHCN

SEQ ID NO: 76 - protein, 152 aa, Artificial Sequence
  AtEIL3
DLERRMWKDRVRLKRIKERQKAGSQGAQTKETPKKISDQAQRKKMSRAQDGILKYMLKYMLKLMEVCKVRGFVYGIIPEKGKPVSGSSDNIRAWWKEKVKFDKNGPAAIAKYEEECLAFGKSDGNRNSQFVLQDLQDATLGSLLSSLMQHCD

SEQ ID NO: 77 - protein, 154 aa, Artificial Sequence
  PsEIL1
ELERRMWKDRIKLIRIKERQKLAAQQAAEKQKPKQTTDQARRKKMSRAQDGILKYMLKYMLKLMEVCKARGFVYGIIPEKGKPVSGASDNIRAWWKEKVKFDKNGPAAIAKYDADCLAMGESESYRNGSSQSVLQDLQDATLGSLLSSLMQHCD

SEQ ID NO: 78 - protein, 104 aa, Artificial Sequence
  MaEIL2p
DGILKYMLKYMLKLMEVCNVRGFVYGIIPEKGKPVSGASDNIRAWWKEKVKFDKNGPSAIAKYEAENFAADKAQNSGSKNQCSLADLQDATLGSLLSSLMQHCD

SEQ ID NO: 79 - protein, 169 aa, Artificial Sequence
  AtEIN3.5
DLKKRMWKDRNLMCKLKQQKRDNLNSVISSPSSSTSASSSSSSSVIVRRTEASRRKKMARSQDSVLKYMMKYMMKIMEVCKAQGFVYGIVPEKGKPVTGSSDSLRRWWKENVQFDQTAPNAVSDYLTLAAAQLISSNESLDPNSYIHMLHELQDTTLGSLLSALMQHCV

SEQ ID NO: 80 - protein, 156 aa, Artificial Sequence
  AtEIL4
DLKRRMWKDRNLMEKKLKQQKRHSNDVVSFTTHRAEASRRKKMARSQDSVLKYMMKYMMKIMEVCKAKGFVYGIVPEKGKPITGSSDSLRRWWKENVQFDQNAPDAITDYLALAAAAAAAELIDKSSSSSSLLHMLQELQDTTLGSLLSALMQHCM

SEQ ID NO: 81 - protein, 60 aa, Artificial Sequence
  C.medium_cultivated.
SNSIGPTPHTLQELQDTTLGLLLSALIVILHRGVSLSRKVFRPRGGPRGRRNGGLNWVCR

SEQ ID NO: 82 - protein, 60 aa, Artificial Sequence; identical to SEQ ID NO: 81
  C.medium_calycanthema.

SEQ ID NO: 83 - protein, 60 aa, Artificial Sequence; identical to SEQ ID NO: 81
  C.medium_wild.

SEQ ID NO: 84 - protein, 60 aa, Artificial Sequence
  C.medium_AP.
SNSIGPTPHTLQELQDTTLDLLLSALIVILYRGVSLSRKVFRPRGGPRGRRNGGLNWVCR

SEQ ID NO: 85 - protein, 60 aa, Artificial Sequence; identical to SEQ ID NO: 84
  C.medium_6.

SEQ ID NO: 86 - protein, 60 aa, Artificial Sequence; identical to SEQ ID NO: 84
  C.medium_80.

SEQ ID NO: 87 - protein, 60 aa, Artificial Sequence; identical to SEQ ID NO: 84
  C.medium_123.

SEQ ID NO: 88 - protein, 60 aa, Artificial Sequence
  C.medium_64.
SNSIGPTPHTLQELQDTTLGSLLSSLIVILPRGVSLWRKVFRPRGCPRGRRNGGLNWVCR

SEQ ID NO: 89 - protein, 60 aa, Artificial Sequence; identical to SEQ ID NO: 88
  C.medium_11.

SEQ ID NO: 90 - protein, 60 aa, Artificial Sequence; identical to SEQ ID NO: 88
  C.medium_3.

SEQ ID NO: 91 - protein, 60 aa, Artificial Sequence; identical to SEQ ID NO: 88
  C.medium_30.

SEQ ID NO: 92 - DNA, 66 bp, Artificial Sequence
  CfEIL2
agagcttcaagatacgaccctcggttcgttattatctgctctcatgcagcattgtgatcctcccca

SEQ ID NO: 93 - DNA, 59 bp, Artificial Sequence
  CmEIL2
agagcttcaagatacgaccctcgatttgttattatctgctctcattgtgatcctctaca